(19) United States
(12) Patent Application Publication
(10) Pub. No.: US 2009/0287282 A1
(43) Pub. Date: Nov. 19, 2009

(54) Thermal bodily compress kits and methods of using same

(76) Inventors: Seth Biser, Fleetwood, NY (US); Lawrence Thad Levine, Easton, CT (US); Harvey Levine, Fairfield, CT (US); William Finneran, New York, NY (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/153,321

(22) Filed: May 16, 2008

(51) Int. Cl. A61F 7/02

(52) U.S. Cl. 607/109

(57) ABSTRACT

A thermally adjustable compress assembly, kit, device and methods of using the same. A compress assembly includes a thermally adjustable gel pack and a plurality of moistened, disposable sheets that are removable from the outer surface of the gel pack. The sheets can be fabricated from a non-woven fabric material, a foam material or a film material. A compress assembly can further include an external frame attachable to the gel pack and one or more sheets of the plurality of sheets.

### CROSS-REFERENCE TO RELATED APPLICATIONS

The present application incorporates by reference U.S. application Ser. No. ______ filed on ______ entitled “THERMAL COMPRESS ASSEMBLY AND SYSTEM WITH EXTERNAL FRAME” (Attorney Docket No. 14414/3).

### TECHNICAL FIELD

The present invention is directed to thermal body compress devices, kits, assemblies, systems, and methods of using the same. In a particular application, the present invention is directed to providing therapeutic benefit to a user's eye region.

### BACKGROUND

Both hot and cold compresses play an important role in treating various physical problems. As an illustrative example, not meant to be limiting, one specific area of such problems relates to ocular discomfort and disease. In this example, eye care practitioners have recommended hot compress therapy for various eye conditions including certain types of dry eye syndrome, “styes,” orbital and preseptal cellulitis, acute dacryocystitis, and other conditions. Hot compresses to the eyelids and periorbita are also used for certain postsurgical states, for the promotion of feelings of relaxation, for certain cosmetic or dermatological treatments, and for various other reasons. Cold or cool compresses have been recommended for postoperative states following eye surgery, for symptomatic relief of irritating conditions, for relief of migraines, to promote feelings of relaxation, to allow the application of topical skin therapies for cosmetic and dermatologic treatments, and for various other reasons. In addition to professionally-encouraged use, user-directed self-administration of both hot and cold compresses has been fairly widespread.

In the most common and traditional method of compress therapy, the user holds a washcloth either under hot or cold running tap water, or in a basin of hot or cold water, and then applies the moist, temperature-adjusted washcloth to the body part. This method is popular because washcloths are low in cost and widely available, they are reasonably soft in texture, and their temperature can usually be determined by the user. In addition, the washcloth method allows the user to select how the external pressure is applied against the body part. The specific case of eye compresses is illustrative. Because the eyes are one of the most sensitive and delicate of bodily tissues, most users of the washcloth method will avoid putting pressure directly on the round globe of the eye (the eyeball), and will instead press the washcloth gently into other areas such as the corners of the eyes. The washcloth thereby passively conforms to the round globe of the eye in a safe and comfortable way. Therefore, the washcloth method has been viewed as being particularly useful for hot compress therapy.

However, the washcloth method has numerous disadvantages. The washcloth's temperature decays relatively quickly necessitating frequent re-heatings or re-coolings, especially if the washcloth is wrung out after immersion in water. In the case of compress therapy applied to the eyes or other specific head regions, the washcloth may drape uncomfortably over the face and, if too wet, will tend to drip down the user's arm as the user stands at the sink. Repeated use on a body part of a washcloth left in a bathroom, especially when the bathroom is shared by more than one person, may be unhygienic.

Other efforts to apply sustained thermal application as a part of compress therapy are also known. One example is a gel pack which can be heated in a microwave oven and applied against the user's body part. An issue with using gel packs on sensitive body parts such as the eyes and periorbital region, however, is the sensitivity of the skin to the physical nature of the outer covering of gel packs. The external layers of most gel packs is composed of a plastic or vinyl surface which is slick and non-wettable. Such materials are commonly perceived as not allowing the skin to “breathe,” and may produce discomfort from prolonged contact. Many users object to the “artificial” and “plastic” feeling these materials impart. Some gel packs, such as that described in U.S. Pat. No. 6,648,909 to Helming, specifically supply some type of fabric that is permanently attached to the gel pack itself. Helming specifically states the need to avoid a water absorbent fabric in this application. However, such non-wettable fabrics are typically not useful for sensitive areas such as the eyes and ocular regions (as well as other regions), where users tend to prefer wettable materials such as washcloths. Furthermore, repeated use of a gel pack that is supplied with a permanently-attached fabric piece will tend to be unhygienic, owing to the difficulty of washing the fabric piece while it is attached to the gel pack. For example, Helming describes a gel pack intended for use in the perineal region, a field of art in which gel packs are typically chemically-activated for one-time use, and intended to be disposable.

While some gel pack devices designed for compress therapy mention that “cloth,” “4×4 gauze pads,” and “paper towels” (which are not attached to the gel pack) may be used in coordination with the gel packs devices, these gel pack devices do not specifically address some of the shortcomings of these materials which the inventors of the present inventions have found (and which are therefore not necessarily recognized in the art). For example, cloth materials can become mildewed between uses unless regularly washed; gauze pads are quite thin and do not provide either good cushioning or moisture retention; and paper towels tend to have a rough surface, do not provide good cushioning, dry out quickly when repeatedly used, and tend to disintegrate when kept wet for long periods.

Further, because the recommendation for these other materials implies that the end-user would be supplying such materials, the inconvenience is displaced onto the user. For example, the user would need to arrange for the selection, sizing, and shaping of the material and may have to launder the fabric for repeated use. In addition, the specific non-cloth materials mentioned previously (such as 4×4 gauze pad and paper towels) do not address the particular needs of a compress system applied to a sensitive body part, in which it would be desirable to supply a material that is soft, cushioning, and is designed to retain moisture for long periods of time without disintegration.

None of the current gel pack system designs provide materials that are specially shaped and moistened for use on the body part that is the target of thermal compress therapy.

### SUMMARY

The present invention provides thermally adjustable body compress devices, assemblies, kits, systems, and methods of preparing and using the same. The body compress assemblies, systems, and kits can be used to provide symptomatic relief of bodily symptoms or to otherwise improve the user's condition.

In an embodiment, the present invention provides a thermal bodily compress assembly and system. The compress assembly and system comprise a thermally-adjustable gel pack that is configured to be applied against a body region of a user's body. The gel pack comprises a casing defining a chamber holding a thermally-activatable gelatinous substance. The compress assembly and system further comprise a strap attached to the casing to secure the gel pack against the patient's body region and to exert compressive force to the gel pack. The compress assembly and system further comprise a moistened, disposable fibrous non-woven fabric sheet passively positionable between the gel pack and the patient's body region to form a body compress system or actively positionable between the gel pack and the patient's body region to form a body compress assembly. The fabric sheet is removable from the outer surface of the gel pack. In other embodiments, the sheet is a foam sheet. In still other embodiments, the sheet is a film sheet. The sheet provides, for example, a hygienic surface for the user; provides a surface that is wettable, unlike the surface of the gel pack, and can thereby aid in thermal therapies such as heat therapies that are believed to improve with the application of moisture; provides a surface that can be impregnated with various medications and other therapeutic substances, unlike the surface of the gel pack; and serves in part as a thermal reservoir to aid in intensifying the thermal therapy to the user. In use, one or more sheet(s) is (are) disposed between the user's face and the gel pack either passively or actively via a fastener to form a compress system or assembly, respectively. The sheets can also be impregnated with a medication or other chemical(s) specific for treating a physiological condition of the body part against which the sheet is placed.

The present invention also provides a thermal body compress kit comprising a gel pack as described above and a plurality of sheets as described above. The plurality of sheets can be contained within a dispenser. The dispenser can be designed for home use or travel use.

In certain embodiments, a compress assembly and system further comprise an external frame attachable to or otherwise positionable against the outwardly facing side of the gel pack (i.e. the side of the gel pack that will not be in contact with the patient's body region in an applied position of the gel pack). In an embodiment, the external frame is attached to or otherwise positioned against the gel pack in use to compress the gel pack against the user's anatomy. In certain embodiments where the external frame is attached to the gel pack, the external frame vertically supports at least a portion of the gravitational weight of the gel pack so that the gel pack does not buckle. Furthermore, in certain embodiments, the top portion of the gel pack is attached to the top portion of the external frame and a strap is attached to the right and left sides of the external frame such that tightening or loosening of the strap exerts a controllable horizontal pressure on the external frame which is largely independent from the vertical support provided by the external frame to the gel pack. Such an embodiment allows, among other things, for the user to keep the thermal compress assembly in contact with the body with only a minimally compressive effect.

In certain embodiments, the external frame defines relief openings that provide relief from the direct compressive pressure that would otherwise be transmitted through the external frame at the locations of the openings. Such openings also allow the user to manipulate the gel pack in areas the gel pack directly underlies in order to achieve a precisely-directed therapeutic effect.

The compress assemblies and systems can be thermally activated by exposure to cold or heat. For example, the compress assemblies and systems could be placed in a refrigerator or freezer, exposed to a cold water or ice bath or exposed to another cold source. The compress assemblies and systems could be exposed to heat by microwave irradiation, a hot water bath or other heat source.

### BRIEF DESCRIPTION OF THE DRAWINGS

FIG. 1 is a schematic illustration in perspective view of an exemplary gel pack according to an embodiment of the present invention.

FIG. 2 is a front view of an exemplary gel pack according to an embodiment of the present invention.

FIG. 3 is a front view of an exemplary gel pack according to an embodiment of the present invention.

FIG. 3A is a front view of an exemplary gel pack according to an embodiment of the present invention.

FIG. 4 is a schematic illustration of an average male human face for purposes of illustrating certain anatomical landmarks and distances therebetween.

FIG. 5 is a front view of an exemplary gel pack according to an embodiment of the present invention.

FIG. 6 is a perspective view of an exemplary gel pack according to an embodiment of the present invention.

FIG. 7 is a front view of an exemplary gel pack according to an embodiment of the present invention.

FIG. 7A is a front view of an exemplary gel pack according to an embodiment of the present invention.

FIG. 8 is a side view illustrating of an exemplary gel pack in a resting position according to an embodiment of the present invention.

FIG. 9A is a side view of a gel pack having a bulging configuration in a resting position.

FIG. 9B is a side view of a gel pack having a bulging configuration in an applied position.

FIG. 10A is a schematic illustration of an assembled eye compress assembly in an applied position according to an embodiment of the present invention.

FIG. 10B is a schematic illustration of an assembled eye compress assembly in an applied position according to an embodiment of the present invention.

FIG. 11 is a front view of an exemplary sheet according to an embodiment of the present invention.

FIG. 12 is a perspective view of a dispenser to hold a plurality of sheets according to an embodiment of the present invention.

FIG. 13 is a perspective view of a dispenser to hold a plurality of sheets according to an embodiment of the present invention.

FIG. 14 is a front view of an external frame according to an embodiment of the present invention.

FIG. 14A is a plan view of an external frame having a generally planar configuration in a resting position.

FIG. 14B is a plan view of an external frame having a concave surface in a resting position.

FIG. 15 is a front view of an external frame according to an embodiment of the present invention.

FIG. 16 is a schematic illustration of an assembled eye compress assembly in an applied position according to an embodiment of the present invention.

FIG. 17 is a front view of an external frame according to an embodiment of the present invention.

FIG. 18A is side view of a fastener that includes a button according to an embodiment of the present invention.

FIG. 18B is a side view of an assembled eye compress assembly using the fastener of FIG. 18A.

FIG. 19 is a side view of a fastener according to an embodiment of the present invention.

FIG. 20 is a front view of an external frame according to an embodiment of the present invention.

FIG. 21 is a front view of an external frame and gel impresser according to an embodiment of the present invention.

FIG. 22 is a graph of the temperature of a gel pack over time using a wet non-woven sheet and a dry non-woven sheet.

FIG. 23 is a graph of the temperature of a gel pack over time using a wet non-woven sheet and a wet terrycloth towel.

### DETAILED DESCRIPTION

The present invention is directed to thermally adjustable body compress devices, assemblies, kits, systems, and methods of preparing and using the same. The devices and methods can be used to treat or alleviate a variety of abnormal physiological conditions in users or to provide therapeutic benefit to users who are otherwise in normal condition. The devices and methods can be applied to various body parts such as, for example, the soft tissues, muscles, bones, and other tissues and organs of a user. Non-limiting examples of anatomical sites that devices and methods can be used for include the knee, ankle, and other parts of the leg; the shoulder; the neck; the ears; the back including the lumbar and cervical regions as well as other areas of the back; the face, including the nose and nasal region, the jaw and eye region; and the perineal region. Although the present invention will be described with relation to applying the compress devices and methods to an eye region of a user, it is understood that the invention has broader application to other parts of the anatomy including those specifically mentioned above. As used herein, the term “user” includes mammalian subjects including humans.

At a minimum, an eye region of a user that is treated by devices and methods of the present invention includes the periocular region. According to the present invention, the periocular region is defined as including the eyelid, including the skin of the upper and lower eyelids; the eyelid margins; and the lateral canthus and the medial canthus. The periocular region can, but is not required to, include the region of skin directly overlying the ethmoid sinus. In other embodiments, the eye region includes the periorbital region. According to the present invention, the periorbital region includes the eyebrow; either or both of at least a portion of the skin overlying the frontal sinus and at least a portion of the skin overlying the maxillary sinus; at least a portion of the upper cheek; the bridge of the nose; and at least a portion of the temple of the head. In certain embodiments, the periorbital region includes the eyebrow, the skin overlying the entire frontal sinus, the skin overlying the entire maxillary sinus, the entire upper check, and the entire temple. In other embodiments, the eye region includes both the periocular region and the periorbital region. Of course, the above described anatomical sites are described in the singular tense but it is understood that these regions are bilateral and thus embodiments of the present invention also cover both the left and right periocular and/or periorbital regions. Of course, in certain embodiments, only the left or right eye region is covered. In other embodiments, the eye region includes the entire temple(s) of the head. In certain embodiments, the eye region includes only one or more of the above-described regions (i.e. does not include the entire face or head).

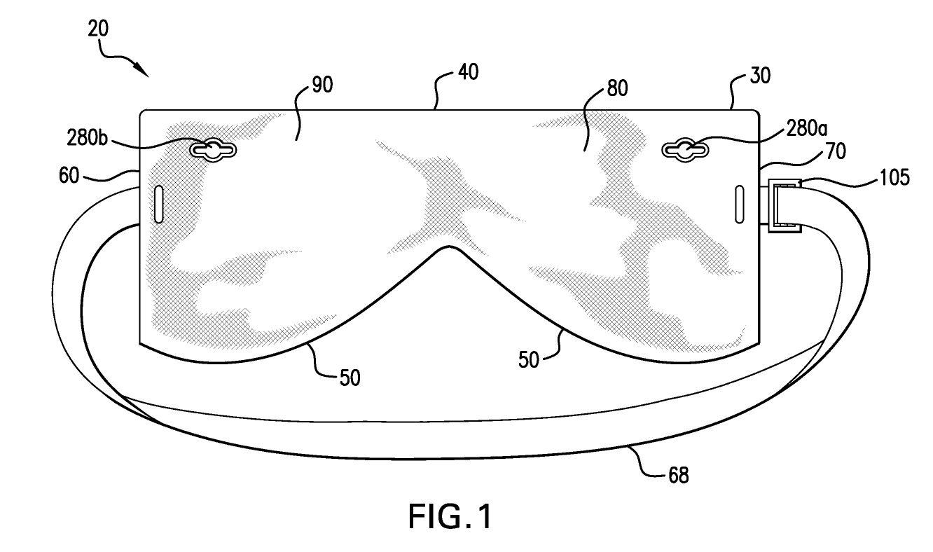

Referring to FIG. 1, in an embodiment, the present invention provides a compress assembly and system comprising a thermally adjustable pack 20 configured to be applied against the eye region of a user's face to apply a sufficient heat or cold source to the user's eye region to provide a therapeutic benefit to the user. By providing a therapeutic benefit to the user, the compress assembly improves a given user's condition compared to the same user's condition prior to use. Accordingly, pack 20 comprises a thermally activatable substance whose temperature can be regulated or adjusted by applying various degrees of heat or cold. Such a substance is capable, at a minimum, of being warmed or cooled so that it achieves a temperature that is substantially different from room temperature, and sustains the achieved temperature for a relatively long period of time and with a relatively slow period of decline back toward room temperature. By sustaining an achieved temperature for a relatively long period of time with a relatively slow period of decline back toward room temperature means that if 2.5 ounces of such a substance were heated from room temperature (72° F.) to a temperature of 135° F. and left in an uncovered condition (without insulation), the substance would maintain a temperature of at least 115° F. for at least five minutes after heating had taken place. Non-limiting examples of thermally activatable substances include water; various gelatinous materials such as solid or semi-solid gels; dried vegetables and cereals such as rice, beans, corn, and peas; water-containing food products such as potatoes and apples; and various other vegetables and food products. In a preferred embodiment, the thermally activatable substance is a gelatinous substance (also referred to herein as a “gel” or “gelatinous material”) and the thermally activatable pack is a gel pack. The below-described embodiments will be described with respect to a gelatinous substance although it is understood that other thermally activatable substances can also be used.

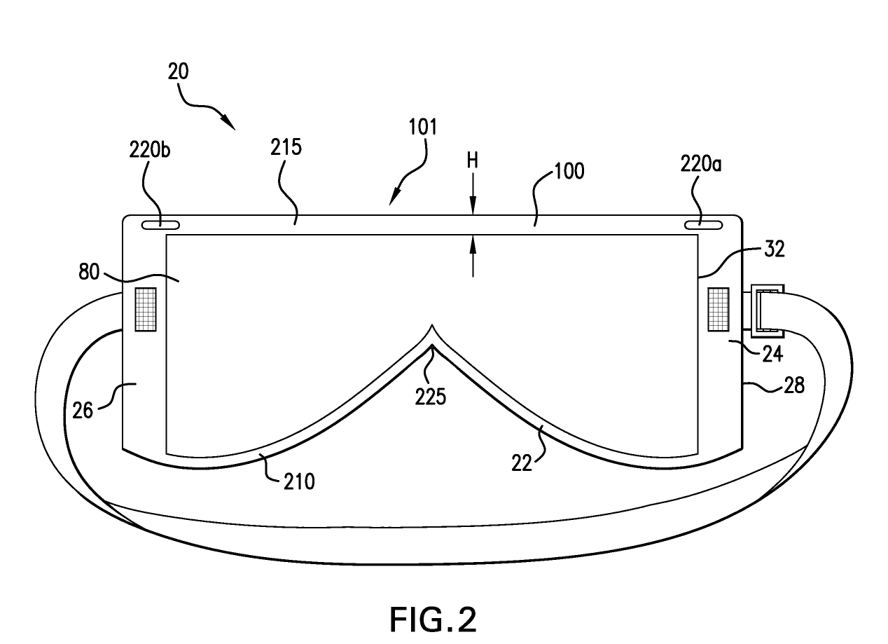

Referring again to FIG. 1, gel pack 20 comprises a casing 30 having a top portion 40, a bottom portion 50, a right portion 60, a left portion 70, a front side and a back side. As used herein in relation to the below description and accompanying figures, the terms “top,” “bottom,” “left,” “right,” “front,” and “back” refer to the orientation of the gel pack and compress assembly and system in relation to the user, in an applied position on the user's face when the user is standing upright (a position known in the art as the “anatomical position”) and facing out of the page toward the viewer. Of course, the gel pack and compress assembly and system can be used either in an upright (sitting or standing) or recumbent position. Reference to the user standing upright is only to provide a standard by which to understand the above-referenced locational terms. The front side of the gel pack is the side that faces outwardly and is the side illustrated in FIG. 1. The back side is the opposite side of the gel pack which faces the user in an applied position of the gel pack (i.e. when the gel pack is in use). Gel pack 20 defines a chamber 80 (illustrated more clearly in FIG. 2) holding a gelatinous thermal substance 90. In certain embodiments, casing 30 comprises at least two layers of flexible sheets sealed about their edges to form chamber 80. In those embodiments, casing 30 has a periphery defined by the sealed edges of the flexible sheets. The periphery of casing 30 can be co-extensive with the periphery of chamber 80 such that there is no space between chamber 80 and casing 30 as seen in FIG. 1. In other embodiments, as shown in FIG. 2, the periphery is divided into a top lip 101, which can further be divided into a top left lip 100 and a top right lip 215; a bottom lip 103, which can further be divided into a bottom left lip 22 and a bottom right lip 210; a left side lip 24; and a right side lip 26, where the lips are the regions between the outermost edge 28 of casing 30 and the outermost edge 32 of chamber 80 (and therefore such lips contain no gelatinous material).

In certain embodiments, top lip 101 has a height sufficient to accommodate fasteners to attach the gel pack to a sheet or to attach a gel pack to a sheet and a support structure (as described in more detail below). Briefly, the support structure can be used to compress the gel pack against the user's anatomy and optionally to vertically support at least a portion of the gravitational weight of the gel pack when the gel pack is in an applied position. In addition or alternatively, left and right lips 24 and 26 have a length sufficient to accommodate such fasteners. In other embodiments, the bottom lip 103 has a height sufficient to accommodate such fasteners. In other words, the periphery of the casing can be sized to accommodate fasteners in various different locations. With specific reference to the embodiment illustrated in FIG. 2, apertures 220, one defined by top left lip 100 and the other defined by top right lip 215 are shown that can receive fasteners, such as buttons, for example, to fasten the gel pack to a sheet(s) or to a sheet(s) and a support structure Alternatively, the top lip 101 of the gel pack can have fasteners attached thereto to secure the gel pack to a sheet(s) or to sheet(s) and a support structure. In certain embodiments, top lip 101 has a height H of between about 2 millimeters (mm) and 20 mm. In a more preferred embodiment, top lip 101 has a height H of between about 10 mm and 15 mm. Of course, as shown in FIG. 1, gel pack 20 can be configured such that the outermost edge of top portion 40 of casing 30 is coextensive with the outermost edge of the top portion (not shown) of chamber 80 and can still accommodate fasteners. For example, the top portion 40 of gel pack 20 in FIG. 1 can define apertures 280 similar to the apertures of FIG. 2, as long as the edges of the apertures are sealed to prevent leakage of the gelatinous substance.

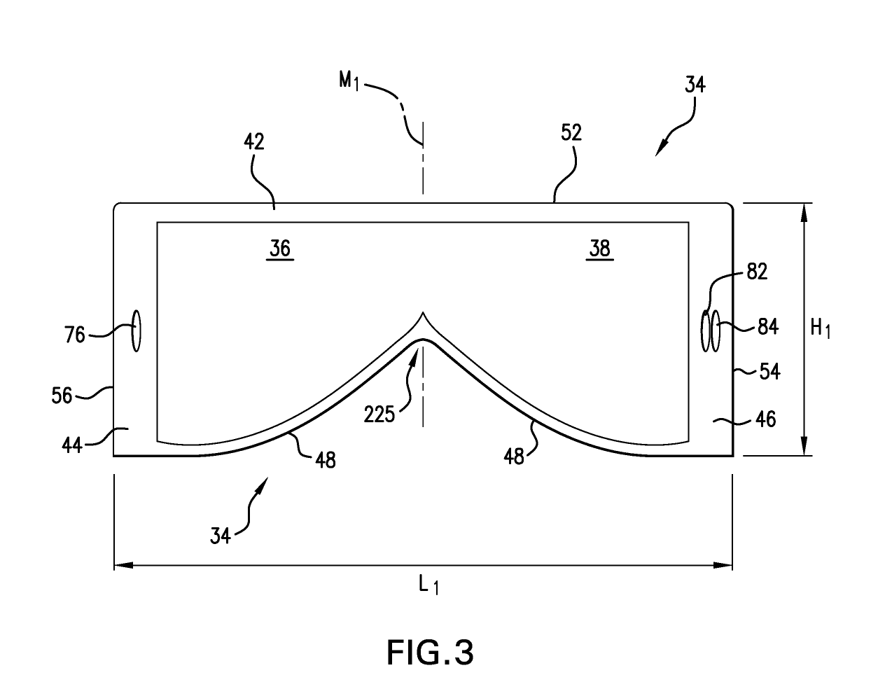

The gel pack can have various configurations. Such configurations can depend, for example, on the body region, such as the eye region, of the user that the gel pack is applied against. For example, referring to FIG. 3, a gel pack can be in the form of a mask 34 that is configured to cover the periocular and periorbital regions of the user's face. As shown in FIG. 3, the mask has a generally rectangular configuration with a substantially triangular notch 225 for the nasal area. Of course, the mask could have other configurations as well such as a generally oblong configuration with a similar cut out to receive the nasal wings. As shown in FIG. 3, the mask has a centerline M₁ dividing the mask 34 into a right section 36 and a left section 38 that spans over both the left and right eyes respectively of the user in an applied position. Specifically, right section 36 is configured to be applied against the right periocular and periorbital region of an average user's face and left section 38 is configured to be applied against the left periocular and periorbital regions of an average user's face. By being configured to be applied to certain eye regions of an average person's face, a gel pack is configured to cover these regions of an average person's face and provide therapeutic benefit to the user. However, while the gel pack is configured to cover certain regions of an average person's face, the gel pack can be used on a user who does not have average facial dimensions but who still can receive therapeutic benefit from a gel pack.

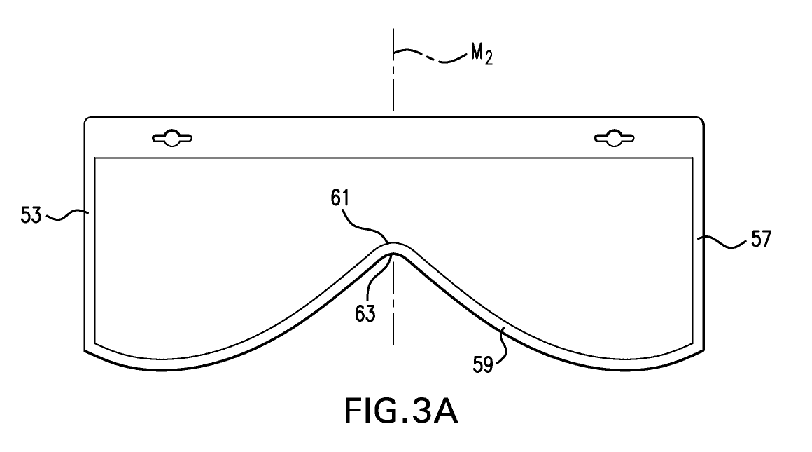

Referring to FIG. 4, which provides a schematic illustration of an “average” adult male face, certain periocular dimensions of the average human face, including children's faces, are as follows: the distance (1) between the inner canthus of the left eye and the inner canthus of the right eye (1) is about 20 to 36 mm; the distance between the center of the right pupil and the center of the left pupil (the interpupillary distance) (2) is about 46 to 75 mm; the distance between the outer canthus of the left eye and the outer canthus of the right eye (3) is about 71 to 105 mm; and the distance between the inner canthus of the left and right eye and the outer canthus of the respective left and right eye (the horizontal palpebral fissure width of each eye) (4) is about 25 to 32 mm. Of course, as mentioned above, other users' facial dimensions may fall slightly out of these ranges but the devices and methods will still be of therapeutic benefit to such users. The above-referenced measurements simply provide a guide to understand the general configuration of the gel packs when used for the eye regions.

Although the left and right sections of a gel pack can be separated from one another such that they are not in fluid communication, in the embodiment shown in FIG. 3, the left and right sections are in fluid communication with each other (i.e. there is no physical separator or divider between the two sections). This feature may be preferred and proved advantageous when the gel pack is activated in a microwave oven and where there is uneven heat distribution applied to the gel pack. Pressure can be applied to the unevenly heated gelatinous substance (i.e. applying back and forth pressure between the two sections of the gel pack) to allow redistribution of the gelatinous substance that resulted in a more homogenous heating effect when in use.

Regarding the specific configuration of a mask that can be used as a gel pack as illustratively shown in FIG. 3, mask 34 has a top portion 42, a right side portion 44, a left side portion 46, and a bottom portion 48. In the embodiment shown in FIG. 3, top portion 42, left side portion 46 and right side portion 44 have relatively straight edges, 52, 54, and 56 respectively but curved edges are also possible. Left and right side portions transition into a bottom portion 48 shaped like a bell curve which essentially creates a notch 225 to accept the nasal wings of the user. Alternatively, the peak of the notch 225 could be angled instead of curved as shown in FIG. 2. Still alternatively, as shown in FIG. 3A, the right lip 53 and the left lip 57 can transition into a bottom lip 59 shaped like a bell curve where both the top 61 of the bell curve shaped section of bottom lip 59 (which center line M₂ passes through) and the bottom 63 of the bell curve shaped section of bottom lip 59 (which center line M₂ also passes through) are both curved.

Referring back to FIG. 3, in certain embodiments, mask 34 has a maximum length L₁, of between about 4 inches and 11 inches. In a preferred embodiment, mask 34 has a maximum length L₁ of between about 5.75 inches and 9.0 inches.

Maximum length L₁ is taken by measuring the length of an imaginary line between the two farthest points on the left and right portions of the mask, the imaginary line being perpendicular to centerline M₁. In certain embodiments, mask 34 has a maximum height H₁ of between about 2 inches and 6 inches. In a preferred embodiment, mask 34 has a maximum height H₁ of between about 2.5 inches and 4.5 inches. Maximum height H₁ is taken by measuring the length of an imaginary line between the two farthest points on the top and bottom portions of the mask, the imaginary line being parallel to centerline M₁.

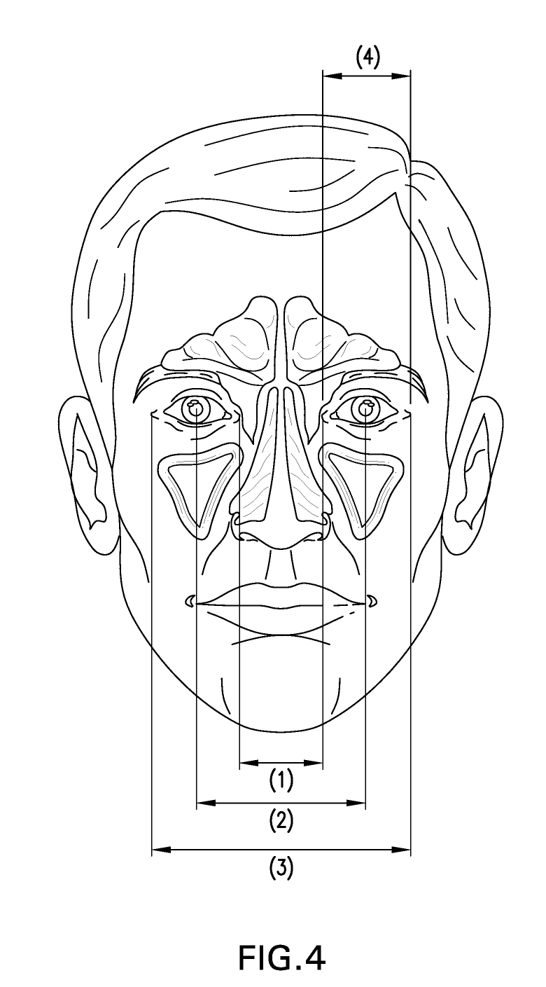

Referring to FIG. 5, in another embodiment, a gel pack is configured similar to the mask of FIG. 3 but with lateral wings 230a and 230b that extend past regions of the left and right side portions respectively of the mask illustrated in FIG. 2 and FIG. 3. Such a configuration may be particularly useful if it is desired to apply thermal compression to the periocular region and the periorbital region, including the temples of the forehead, for example. In other embodiments, the width of the mask could be reduced so that thermal compression is not applied to the entire periorbital region but to the periocular region and the temples of the forehead. In certain embodiments, the mask 34 in FIG. 5 has a maximum length L₂ of between about 6 and 12 inches. The maximum length L₂ of mask 240 is calculated in the same way as the maximum length L₁ of mask 34 is measured as described above with reference to FIG. 3.

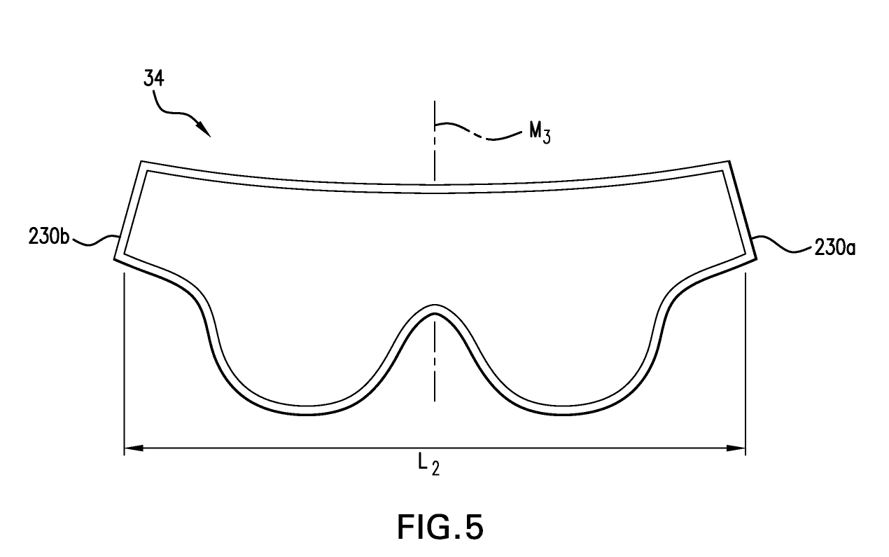

Referring to FIG. 6, in other embodiments, a gel pack can be in the form of goggles 62 which comprise two ring-shaped eyepieces 64 (a left and a right eyepiece) joined together by a longitudinal bridging member 66 that connects the two eyepieces. Bridging member 66 is adapted to bridge the nose of the user. Such an embodiment may be desired if only the immediate periorbital region of the user's face, and not the eyelids, is desired to be covered. As can be seen from FIG. 6, in this embodiment, eyepieces 64 define two apertures 240a and 240b which are substantially centrally located in the eyepieces and are sized to accommodate an average user's eyes. These apertures prevent any gelatinous material from being pressed against the eyeball of the user. In other embodiments, there are no apertures but it is preferable that the volume of gelatinous material near the center of the eyepieces is an amount that will not provide discomfort to globes of the user's eyes (i.e the volume of gelatinous material in the center of each eye piece is less than the volume of gelatinous material in the area of the eyepiece surrounding the center). In certain embodiments, each eyepiece has a circumference of between about 9.0 inches and 12.5 inches. Of course other configurations of eyepieces 64 are also contemplated such as rectangular or oval, for example.

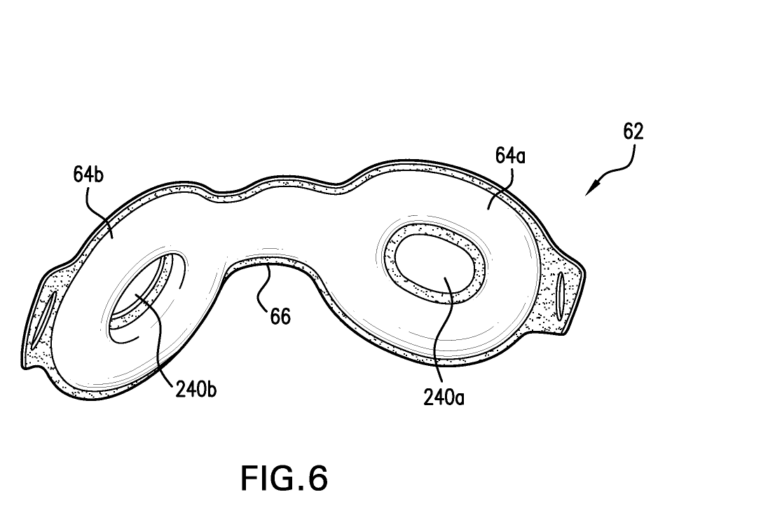

Referring to FIG. 7, in other embodiments, a gel pack is in the form of an eye patch 72 which is configured to cover only one eye region of the user (i.e. either the left or the right eye region). Such a configuration may be useful where therapy is desired for only one eye region. In the embodiment shown in FIG. 7, eye patch 72 has an oval shape but other shapes are also possible such as rectangular or circular, for example. The exemplary dimensions of eye patch 72 can be the same as a single eyepiece of goggles 62 (as shown in FIG. 6) particularly, but not exclusively, when it is desired for the gel pack to cover only one side of the periorbital region of the user's face. Alternatively, as shown in FIG. 7A, the exemplary configuration of eye patch 73 can be substantially similar to a single section of mask 34 as shown in FIG. 3, particularly, but not exclusively, if it is desired for the gel pack to cover only one side of the periocular and periorbital regions of the user's face. In the embodiment shown in FIG. 7A, the gel pack 73 defines apertures 77a and 77b on the top portion thereon to receive fasteners such as button or snaps, for example, to attach to a support structure as described in more detail below. Of course, the top portion can include other types of fasteners to secure itself to a support structure.

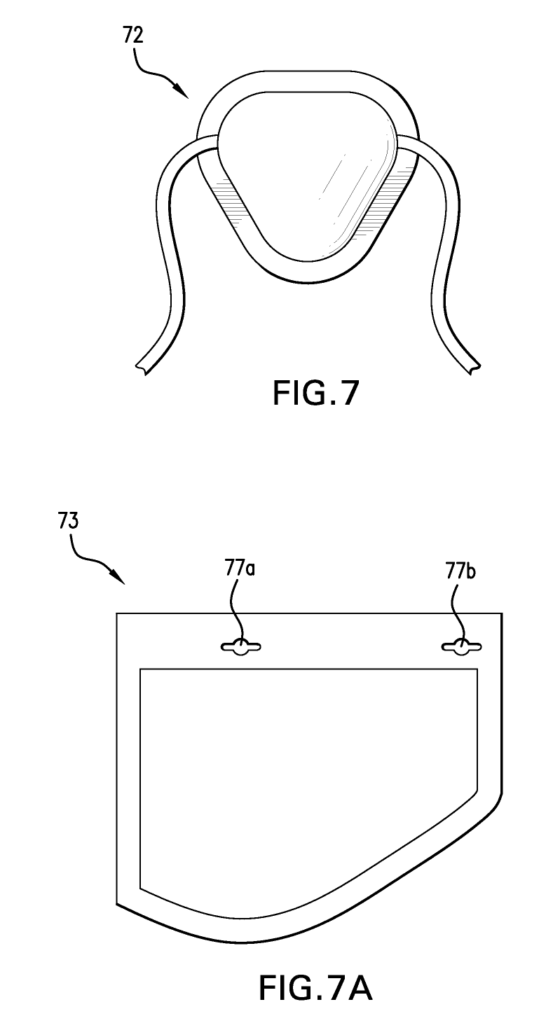

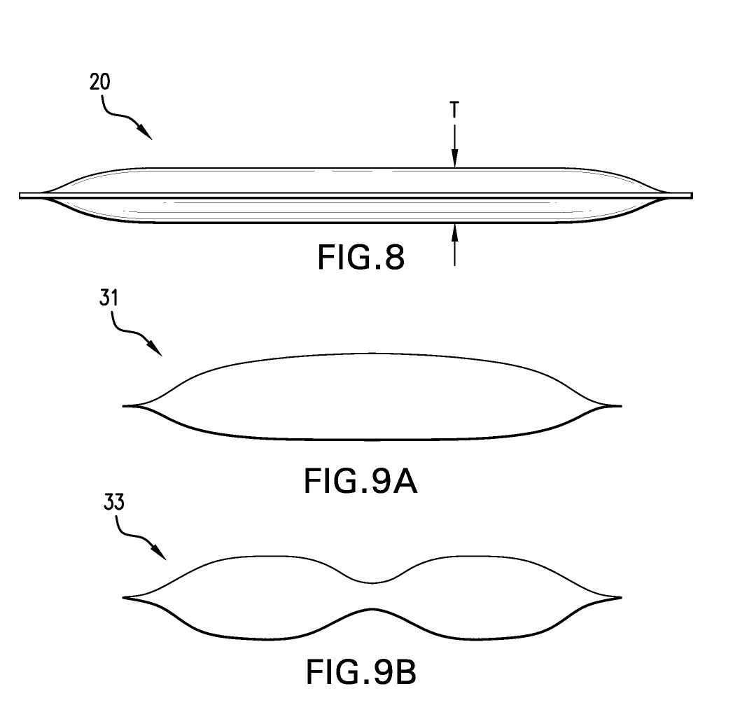

Referring to FIG. 8, in certain embodiments, gel pack 20 has a maximum thickness T between about 0.25 inches and 0.35 inches as taken from the center of the gel pack in a resting position when the gel pack has not been subject to manipulation and the gel is substantially evenly distributed throughout the pack. In certain embodiments, the maximum thickness T of the gel pack does not exceed 0.8 inches so that the gel pack does not have a bulging configuration in a resting position as gel pack 31 does as shown in FIG. 9A or a bulging configuration in an applied position as gel pack 33 does as shown in FIG. 9B. Thus, in a planar configuration, when the gel pack is applied against the eye region of the user, the casing achieves a relatively flat and deformable configuration when it contacts the desired eye regions (i.e. the periocular region or periorbital region of the face), rather than pressing with a bulgingly convex surface against the relative convexity of some of the user's anatomy (such as the globes of the eyes).

Because the gelatinous substance is slippery and difficult to control, a casing is used to contain the gelatinous substance so that the user does not come in contact with the gelatinous substance. The casing can be fabricated from any suitable material to hold the gelatinous substance and to allow thermal diffusion (that is, ready conductivity of heat or cold to the skin, when the gel pack is placed directly or indirectly against the skin). Preferably, the casing of the gel pack is fabricated from any suitable material that can withstand repeated exposure to heat and cool with minimal deformation and without significant degradation. By “minimal deformation” is meant that the gel pack maintains a configuration after 100 heating cycles (with exposure to temperatures between about 100° F. to 160° F.) and/or cooling cycles (with exposure to temperatures between about 40° F. to 0° F.) that is similar enough to its configuration before first use such that it can still perform its intended function and provide therapeutic benefit to the user. By “significant degradation” is meant that the casing degrades to the point that it can no longer perform its intended function and provide therapeutic benefit to a user after 100 heating cycles and/or cooling cycles (at the range of temperatures indicated above).

A preferred material is one that is also flexible enough such that it can sufficiently conform to and be in direct contact with the desired eye regions of the user. The material should also preferably be resistant to any negative chemical effects of the gelatinous substance. Preferably, the material of the casing is waterproof to protect the casing from exposure to moisture (such as in the case of the gel pack being used in conjunction with moistened sheets as described in more detail below). To reduce the chance of microbial buildup with repeated use, materials that can be cleaned with soap and water or alcohol pads are preferred.

Non-limiting examples of materials for the casing including thermoplastic polymers films such as polyamides, polyolefins, and suitable combinations thereof. In a preferred embodiment, the casing is not fabricated from a vinyl material.

Preferably, the periphery of the casing is fabricated from a material of sufficient strength to resist rupture under normal use. However, it may be preferable to allow such rupture in a controlled manner when the chamber pressure is raised to a dangerously high level, such as when a gel pack is inadvertently microwaved for an excessively long time. In such circumstances, slow leakage of contents through a deliberately-ruptured seal would be preferable to explosion of the gel pack. To allow for this slow leakage of contents, the final heat-sealing of the periphery of the casing following gel insertion can be adjusted such that the final heat seal is weaker and less able to withstand an increased internal pressure (as from expansion of water vapor volume of the gel pack chamber during heating) than the material of the casing itself.

As described above, a gel pack includes a chamber that holds a gelatinous substance. The gelatinous thermal substance has characteristics that allow it be malleable enough to conform to the external contour of the user's eye region and to act as an effective thermal reservoir. Specifically, the gelatinous substance preferably comprises a readily deformable gel that can be repeatedly heated and cooled (including freezing) with no appreciable decrease in performance over time. Such heating includes microwaving the gelatinous substance or exposing the gelatinous substance to hot water at temperatures ranging from about 100° F. to 212° F. Such cooling includes placing the gel pack on ice (for example in an ice bath) or within a source of cold air such as a freezer or refrigerator at temperatures ranging from about 40° F. to 0° F. Further, the gelatinous substance preferably comprises a gel that can maintain a desired range of viscosities when subjected to the range of temperatures a user may select. Some stiffening of the gelatinous material would be expected at very low temperatures and some softening at very higher temperatures but the parameters of the gelatinous substance should be preferably such that the substance remains malleable enough so that the user can manipulate the gelatinous substance to maintain its position in a specific eye region for at least 5 minutes. In preferred embodiments, the gelatinous substance can be heated or cooled at least 100 times (at heating temperatures ranging from about 100° F. to 160° F. and cooling temperatures ranging from about 60° F. to 40° F.) while still maintaining its intended function and providing therapeutic benefit to the user.

Parameters of the gelatinous substance that allow for the maintenance of such intended functions include, for example, the composition of the gelatinous substance, the volume of the gelatinous substance, the surface area of the casing, and/or the viscosity of the gelatinous substance. Regarding the composition of the gelatinous substance, non-limiting examples of gelatinous substances include the gelation of xanthan gum, locust bean gum, gum tragacanth, and guar gum; hydroxypropyl cellulose, absorbent and superabsorbent polymers including CARBOPOL™, carboxymethyl cellulose, sodium polyacrylate; similar materials; and suitable combinations thereof.

Regarding the volume of the gelatinous substance, the volume of the gel should provide a sufficiently large mass to serve as an effective thermal reservoir yet not cause the gel pack to bulge and transmit excessive pressure on the eyeball. Of course, in part, the volume of the gelatinous substance depends on the surface area of the casing. For example, if the surface area of the chamber (which is the portion of the gel pack excluding the lips in embodiments where the casing has a peripheral lip as shown, for example, in FIG. 3A) is between 17 to 18 square inches, the volume of the gel is preferably between 2 to 4 ounces. Alternately, if the surface area of the chamber is between 21 and 22.5 square inches, the volume of gel is preferably between 4 to 5.5 ounces. Of course, other dimensions and gel volumes could be used.

The relationship between the amount of gel and the volume of the chamber within the gel pack could be modified to produce gel packs of different sizes and weights, and with different surface characteristics. Gel packs in which the ratio of gel to chamber volume is relatively low would tend to produce packs in which there is relatively little bulging of the surface, and therefore little pressure against the globes of the eyes, but in which the thermal effect of the gel pack is somewhat limited in duration owing to the relatively low volume of gel. Conversely, gel packs in which the ratio of gel to chamber volume is relatively high would tend to produce packs in which there is somewhat more of a bulging contour, and hence somewhat more pressure against the globes of the eyes, but in which there is a more lasting thermal effect owing to the larger volume of gel.

It is desirable that any combination of mass and distribution of gel be sufficient to provide adequate treatment as a thermal compress for a standard duration of treatment. As an example, not meant to be limiting, when the surface area of the chamber is 17.5 square inches and the gel mass is 2.5 ounces, once the gel is heated to 140° F. under experimental conditions, the gel pack is of sufficient mass and has characteristics sufficient to provide a sustained thermal effect for five minutes, such that at the end of five minutes at room temperature, the temperature of the gel pack remains above 110° F.

Of course, the above-described volume and surface areas are only exemplary and the volume and surface area of the respective gelatinous material and casing can be controlled for other configurations of the gel pack in order to achieve a relatively flatter and less bulging contour with a lower gel weight, or a relatively more bulging contour with a higher gel weight.

In the particular instance in which the user desires to employ a gel pack with a larger volume of gel, the user may find it useful to adjust the body part so that gravity pulls the gel pack away from, rather than toward, the body part. In the example of an eye compress, if the user tilts his or her head forward, the gel pack will be positioned away from the eyes, and would therefore spare the user excess pressure on the eyeballs. Doing so would allow the user to employ a heavier gel pack, with a longer thermal effect, without ocular discomfort.

Thus, one way of increasing the duration of thermal treatment is to increase the volume of gel within the gel pack, either by using more gel in a pack of a given volume, and/or by increasing the volume of the pack.

Another possible way of increasing the duration of thermal treatment is by using two or more lower-volume and more planar gel packs, one stacked behind the other. Using lower-volume gel packs in this manner would tend to lower manufacturing, distribution, and sales costs (since only one size and shape of gel pack would be produced), and would tend to give users greater choice during each treatment (allowing each user to select, during a given treatment, whether to use one or more gel packs). This method of stacking gel packs is not previously mentioned in the art, presumably because the challenge of providing a gel pack that is of sufficient volume to sustain a desired hot or cold temperature range, but is also of minimal enough volume that it will not place undue pressure on a sensitive body part such as the eyes, has not previously been effectively addressed.

In general, a lower gel volume-to-chamber area ratio will allow more manipulation of the gelatinous substance so that the user can manipulate the gelatinous substance more freely, and press it into better conformation against his or her own anatomy by creating small bulges in one location and small depressions in another. Preferably the viscosity and volume of the gelatinous substance is such that it allows the user to tailor the amount of gelatinous substance applied against certain eye regions. For example, if the user prefers that a greater thermal effect be applied against a portion of the maxillary sinus, then the user can shape the gelatinous substance such that the gel is easily pressed inward against the region of the face overlying the maxillary sinus. In addition, once deformed to its new inward configuration, the gelatinous substance can be stiff enough so that it will tend to hold its shape for at least 5 minutes relative to this new configuration and will not flow back to its previous configuration by force of gravity.

Ideally and in a preferred embodiment, the material of the casing of the gel pack will work in a synergistic manner with the gelatinous substance contained within it in order to produce a desired outcome of conformation to the user's body part. For example, once the gel is deformed to a desired shape by the user, the inherent stiffness and shape of the casing of the gel pack should preferentially support the shape of the gel in its desired configuration, and help it to resist flowing downward by force of gravity.

Preferably the gelatinous substance has a high water content, allowing rapid energizing by microwave radiation as well as prolonged heat retention due to water's high specific heat. Preferably, the gelatinous substance is biocompatible and non-toxic although it is not expected that a user would come into direct contact with the gelatinous substance during use. Additives could be used to raise the boiling point of the gelatinous substance thereby reducing the risk of vapor production and gas expansion during heating, which could, with prolonged microwave heating, cause the gel pack to burst. Non-limiting examples of such additives include polyethylene glycol. Other additives can also reduce the freezing point, allowing the gelatinous substance to attain low temperatures while maintaining softness and deformability. Non-limiting examples of such additives include sodium chloride. The gelatinous substance could be prepared and sealed in the pack under vacuum conditions in order to minimize the presence of air, thereby further reducing the risk of gas expansion during heating. Lowering the presence of air or gas could also allow for more uniform heating of the gel.

Referring back to FIG. 1, a compress assembly of the present invention can further comprise a strap 68 attached to casing 30 to secure gel pack 20 against the body region of the user, (which in the embodiment shown in FIG. 1 is the eye region) and to exert a compressive force to the gel pack. The strap can be made of any material sufficient to perform these functions. For example, the strap can be fabricated from an elastic stretchable material. Alternatively, the strap can be a non-stretchable material such as a string or ribbon which can be tied to secure the gel pack to the user's face. Preferably, the strap is adjustable allowing the user to exert variable degrees of compressive force to the gel pack. For example, an elastic strap can include a buckle 105 to adjust the tension of the strap according to not only the circumference of the user's head but also according to the degree of compression desired to be applied against the user's eye region. In embodiments where the strap is non-elastic (such as, for example, a string or ribbon), the strap can be tightened by pulling on the ends of the strap to control the compressive function of the strap. Of course, other materials and configurations of strap 68 could also be used. The strap can be made from the same or different material as the gel pack. Referring to FIG. 2, the strap can be attached to the left side lip 24 and the right side lip 26 of casing 30. Of course strap 68 can be attached to other portions of casing 30 so long as strap 68 performs its intended function. In FIG. 2, strap 68 is threaded through slit 76 (illustrated in FIG. 3) of casing 30 and secured to gel pack 20 via an interference fit with slits 82 and 84 of casing 30 (again illustrated in FIG. 3). However, other means of attaching strap 68 could also be used. For example, the strap could be glued or stitched onto casing 30. Alternatively, the strap can be integral with the casing such that the casing and strap are made from the same material and are one-piece in the sense that the strap is not separable from the casing using a normal amount of force without damaging the integrity (i.e. tearing) either the strap and/or the casing. Therefore, strap 68 can be removably or permanently affixed to the casing. Non-limiting examples of material from which strap 68 can be fabricated from include fabrics, plastics, woven elastics, and certain pliable elastic polymers.

It should be noted that while the present invention is described with respect to a single gel pack, more than one gel pack can be used (i.e. stacked against the user's body part).

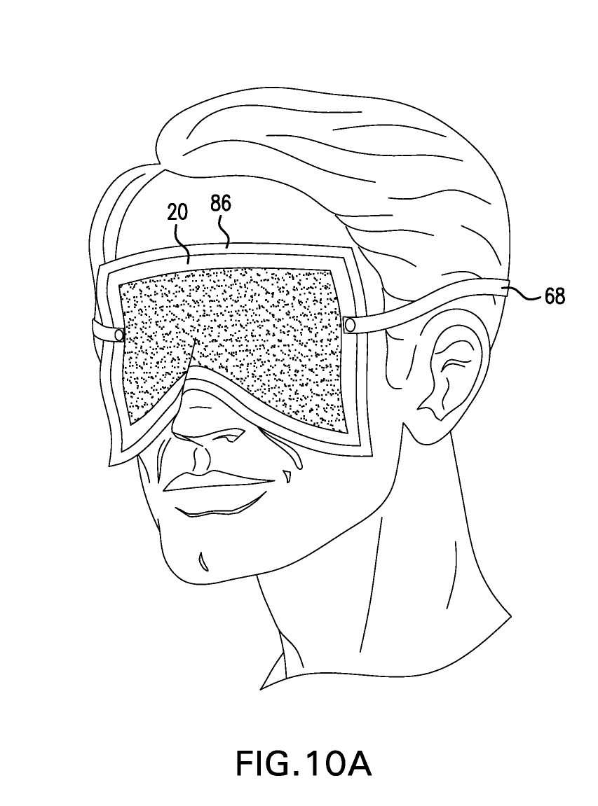

The present invention also provides a body compress system and assembly that includes a gel pack and a sheet removably disposed on the back side of the gel pack. In the exemplary description described above, the body region is the eye region in which case the sheet may be referred to as a “facial sheet.” The sheet serves to provide a wettable cushion between the gel pack container and the user's skin, which cushion can in part serve as a thermal reservoir, but can also serve as a thermal barrier in certain embodiments. The sheet can be passively disposed on the back side of the gel pack to form an eye compress system, in which case the sheet is not removably attached to the gel pack via any mechanical means in a resting position. Instead, as shown in FIG. 10A, sheet 86 is held in place during use by being sandwiched between the user's face and gel pack 20, the latter of which is secured to the user's face via strap 68 that is positioned about the user's head. Alternatively, as seen in FIG. 10B, the sheet can be actively disposed on the back side of the gel pack to form an eye compress assembly in which case the sheet is removably attached to the gel pack via physical means such as at least one fastener 27, which in the illustrated embodiment is a clip, fastens the two components together. Of course, other fasteners could be used including, for example, a male/female fastener, a button, velcro, a magnetic strip, string, or a snap. The fastener can be removably attached to the gel pack as a separable component or can be permanently attached the back side of the gel pack (such as in the case of a button for example) such that the fastener cannot be removed from the gel pack using a normal amount of force without disrupting the integrity of the fastener and/or the gel pack. In embodiments, when an external support structure (discussed in detail below) is also used, the front side of the gel pack may include the same fastener(s) or different fastener(s) as on the back side to attach the gel pack to the front side of the external support structure. At least one fastener can be located on different locations on the back and optionally front side of the gel pack so long as the gel pack can secure the sheet (and the optional external support structure) thereto.

Preferably, the sheet used in the compress assembly and system is moistened, disposable, and/or removably positionable between the gel pack and the body region (in this case the eye region) of the user. By “disposable” is meant that a sheet is designed to be used for a small number of cooling and/or heating cycles and then discarded. Specifically, the same sheet is designed to be heated and/or cooled for a maximum of ten times (i.e. ten uses) before being discarded. In a preferred embodiment, a sheet is intended for a single use after which the sheet is discarded.

By “removable,” “removably positioned” or “removably positionable” is meant that in an applied position, a sheet is not integrally, permanently attached to the gel pack. Thus, a sheet can be removed using a normal amount of force from the back side of the gel pack without disrupting the integrity (i.e. tearing) the gel pack and/or the sheet.

The disposability and removability of the sheet allows for the provision of a fresh and hygienic surface when the user decides to change the sheet (either at every use, or after ten or fewer uses). Frequent exchanges of used sheets with fresh sheets may minimize the risk of infection when re-using the compress assembly or system. The use of new sheets may be especially important when sharing the compress assembly or system with another person. Antimicrobial agents and/or preservatives can be added to the sheet and can aid with prevention of bacterial buildup. The removability and disposability of sheets also provides a more economical method of use, with the relatively inexpensive sheets being replaced after a small number of uses, while the relatively more expensive gel pack can be reused multiple times. The use of removable sheets may also allow the user to choose from a variety of types of pre-medicated sheets, according to his or her needs, during each therapeutic treatment session.

The use of sheets, particularly non-woven sheets, with gel packs in the past has been described in a way that suggests the sheets are not separable or removable from the gel packs under ordinary use, but rather permanently attached to, and an integral part of, the gel packs. For example, U.S. Pat. No. 6,648,909 to Helming describes a perineal thermal pack with a non-woven sheet, which is described as a shell that forms a part of the outer covering for the thermal pack, and is thus inseparable from the thermal pack during normal use. The inseparable relationship between the non-woven sheet and the perineal thermal pack is relevant to the use of the device, as it suggests that the gel pack itself is preferably disposable rather than reusable. In practice, many widely-available perineal thermal gel pack devices are activated chemically for instant use, and marketed for disposable one-time use because of their application to potentially unhygienic areas of the body.

Although the sheets can be dry, in preferred embodiments, the sheet is moistened. More preferably the sheet is pre-moistened such that the user need not moisten the sheet before use. In embodiments where the sheet is moistened, a preferred sheet material is water-absorbent and resilient enough to withstand long periods in a moistened state between the time of manufacture and the time of use without disintegrating. Such a material would also be expected, in its moistened state, to be subjected to manipulation and pulling without significantly tearing or deforming. For instance, a sheet preferably can be subjected to the normal amount of manipulation and pulling necessary to adjust the sheet in relation to the gel pack and optionally with respect to an external support structure (as described in more detail below) during a single use period, which can last between about 2 minutes and 30 minutes. Such manipulation might include repeatedly attaching and detaching a sheet from the external support structure. The preferred sheet material should retain moisture reasonably well, rather than display rapid evaporation, so that users may benefit from a prolonged application of the moist thermal effect. For example, once removed from a dispenser and applied against the user's body region, the sheet material preferably retains at least 60% and more preferably at least 70% and even more preferably at least 80% of its moisture content for at least a 5 minute period of time.

In embodiments where the sheet is moistened, the sheet can be impregnated with various chemicals that may serve a purpose in thermal compress therapy for a particular body part. For example, an eye compress could contain chemicals such as, but not limited to, water, moisturizers, humectants, emollients, nutrifying agents, surfactants, detergents, cleansers, neutraceutical formulations, fragrances and aromatherapeutic compounds, antimicrobial and anti-parasitic compounds, preservatives and buffers, and/or other agents. Specifically, for ocular use, certain chemicals can be selected that may be generally therapeutic for ocular conditions, such as surfactants and humectants that are complementary to molecules normally produced on or near the eyes, as well as chemicals that are therapeutic in specific ocular uses, such as antihistamines, mast cell stabilizers, antibiotics, antiparasitics, corticosteroids, immunomodulatory agents, antiviral agents, and other medications.

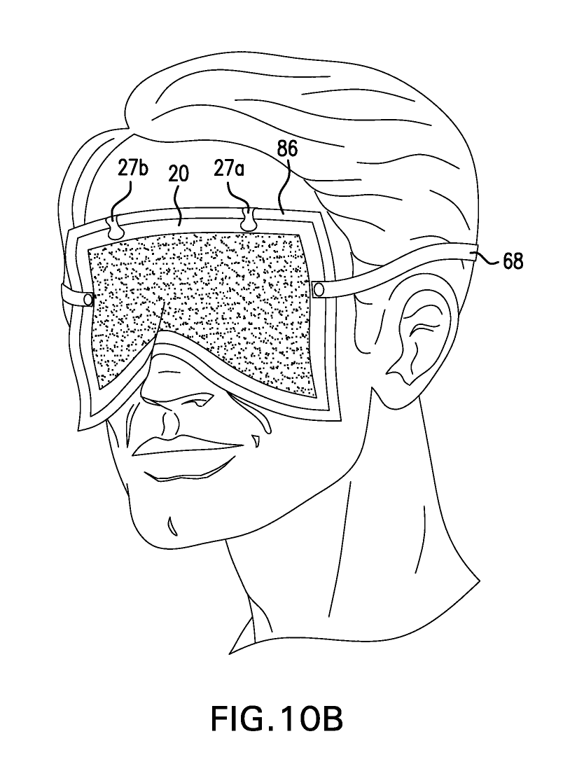

Referring to FIG. 11, an exemplary facial sheet 86 according to an embodiment of an eye compress assembly and system comprises a sheet body 88 that has a top portion 92, a left side portion 94, a right side portion 96 and a bottom portion 98. In the embodiment shown in FIG. 8, top portion 92, left side portion 94 and right side portion 96 have relatively straight edges 104, 106 and 108 respectively but curved edges are also possible. In the embodiment shown in FIG. 11, the right and left side portions transition into a bottom portion shaped like a bell curve which essentially creates a curved cut out 102 to accept the nasal wings of the user. Alternatively, the peak of the cut out could be angled instead of curved (similar to the notch 225 of gel pack 20 shown in FIG. 2). Although the facial sheet 86 shown in FIG. 11 has a substantially rectangular shape with a substantially triangular cut out, sheet 86 can have other configurations such a generally oblong configuration with a similar cut out to receive the nasal wings of the user. In fact, preferably, sheet 86 mimics the outline of the gel pack that is used with the sheet as part of the eye compress kit. However, preferably, a sheet is sized and shaped to extend beyond the edges of the gel pack on all sides so that the user's face is contacted in all applied areas by the facial sheet rather than directly by the gel pack.

In certain embodiments, sheet 86 has a maximum length L₃, of between about 5 inches and 11 inches. In a preferred embodiment, sheet 86 has a maximum length L₃ of between about 7 inches and 9 inches. Maximum length L₃ is taken by measuring the length of an imaginary line between the two farthest points on the left and right portions of the sheet, the imaginary line being perpendicular to centerline M₄. In certain embodiments, sheet 86 has a maximum height H₂ of between about 2 inches and 6.5 inches. In a preferred embodiment, sheet 86 has a maximum height H₂ of between about 3 inches and 4.75 inches. Maximum height H₂ is taken by measuring the length of an imaginary line between the two farthest points on the top and bottom portions of the sheet, the imaginary line being parallel to centerline M₄.

As shown in FIG. 11, in certain embodiments, top portion 92 of sheet body 88 defines openings 110 to accommodate fasteners to attach a sheet to a gel pack or to attach a sheet to a gel pack and a support structure (described in more detail below). The support structure can be used to vertically support at least a portion of the gravitational weight of a sheet when the sheet is in use (in an applied position). The openings can be defined in different locations of sheet body 88 as described above with respect to gel pack 20. Similarly, as described above with respect to a gel pack, the sheet body can have fasteners attached thereto to secure a gel pack or a gel pack and an external support structure.

A sheet can be fabricated from a suitable biocompatible material. A preferred sheet material is preferentially soft in texture, thereby exposing the user's skin to a surface that is more comfortable than the slick, non-moist casing of the gel pack. A preferred sheet material will also have a slight cushioning effect to reduce the impact of the gel pack against the user. A preferred sheet will sustain its integrity after being stored in a moistened state for up to several months, and will be resilient enough to resist tearing or ripping when attached to fasteners that removably affix it to the surface of the gel pack. A preferred sheet material may also allow gentle wiping of the skin. Following the final use of a given sheet, the sheet itself could be used to cleanse the skin of the body part being treated. In the example of an eye compress assembly, a facial sheet could be used to clean debris, oil, crusts, and moisture from the eyelids, as well as to wipe any residual moisture from the skin left there as a result of use of the wet compress.

A sheet can be fabricated from a variety of materials to perform its intended functions. Non-limiting materials include woven or knitted fabrics, non-woven fibrous fabrics, films and foams.

As used herein, the term “non-woven fabric” means an assembly of fibers held together by means and/or processes other than those used in traditional weaving processes. Processes used in the creation of non-woven fabrics include, but are not limited to, mechanical interlocking in a random web or mat, thermal fusing of fibers, or bonding with a cementing medium such as starch, glue, casein, rubber, latex, or one of the cellulose derivatives or synthetic resins.

The non-woven fabric can be prepared from fibers of any fibrous or fiber forming polymer. Synthetic fiber forming materials can be made from the polymers of classes which include, but are not limited to, polyolefin, polycarbonate, polyacrylate, polymethacrylate, polyester, polyamide, polyaramide, polypropylene, polyurethane and the like, as well as copolymers of the above materials. Modified natural polymers such as but not limited to regenerated cellulose and chitin can also be used. Additionally, natural polymeric fibers can be used which include, but are not limited to, cotton, jute, ramie, hemp, other forms of cellulose and forms of chitin. However, according to the present invention, a non-woven fabric does not include a paper towel. The non-woven fabric can be prepared by techniques including, but not limited to spunbonding, melt blowing, hydro-entangling, hydro-lacing, electrostatic spinning, needling, felting, wet laying and the like.

As used herein, the term “film” means a continuous solid or apertured, perforated or porous sheet which can be formed by many known processes including, but not limited to, extrusion, solution casting, calendaring or slitting. The film can be prepared from any film forming polymer, the classes of which include, but are not limited to polyolefin, polycarbonate, polyacrylate, polymethacrylate, polyester, polyamide, polyaramide, polyurethane and the like, as well as copolymers of the above materials.

As used herein, the term “foam” means a flexible or rigid reticulated sheet. These reticulated foams may be made of open or closed cells. The reticulated foam can be prepared from any foamable polymer, the classes of which include, but are not limited to polyolefin, polycarbonate, polyacrylate, polymethacrylate, polyester, polyamide, polyaramide, polyurethane and the like, as well as copolymers of the above materials. These reticulated foams can be prepared from, but are not limited to preparation from, polymers with internal blowing agents, by addition of blowing agents or by agitation to entrain air or another gas.

In a preferred embodiment, a sheet is a non-woven fabric sheet (which, as described above, excludes a paper towel). In a more preferred embodiment, a sheet is a moistened non-woven fabric sheet. In an even more preferred embodiment, a sheet is a pre-moistened, non-woven fabric sheet. Regarding the latter, a pre-moistened non-woven fabric sheet may be preferred as the amount of user-supplied moisture may tend to be non-uniform between uses, thereby producing unpredictable heating from one use to the next. In contrast to user-moistened fabrics (such as cloth towels including terry cloth towels), removable non-woven fabric sheets can be easily packaged together and pre-moistened in such a way that each sheet taken from the package will contain a relatively predictable amount of moisture. This established amount of moisture may produce a more predictable and therefore safer result when a sheet is treated with a given amount of heat. In particular, this established amount of moisture may produce a more predictable and therefore safer result when the sheet is treated with a given amount of microwave irradiation as a means of heating the sheet (with or without a gel pack).

Non-woven moistened fabric sheets are preferred despite such sheets being critically dismissed in the art as part of a thermal compress assembly. For example, U.S. Pat. No. 6,648,909 to Helming specifically suggests that any non-woven sheets used with a perineal thermal gel pack applied in postpartum states be preferably fabricated of a non water absorbent material, the reason being that “the fluid absorbed in the material will tend to act as an insulator against the cold or heat therapy and will reduce the effectiveness of the device.”

Non-woven sheets in both dry and wet preparations are commonly available to consumers and marketed as cleaning products. Examples include SWIFFER® Sweeper Dry Cloths and various similar dry cloths sold under store brand names which are used to trap dirt and dust and clean soiled surfaces (for example, the SWIFFER® Sweeper appeals to consumers by marketing the product with the logo “Just trap dirt and toss it away!” A related SWIFFER® Sweeper Wet Cloth is marketed to “Clean tough soils and dried messes”). Dirt-trapping and the cleaning of tough soils are not attributes that lend themselves to eye compress therapy.

Despite the teachings in the art that point away from using non-woven moistened sheets, experimental use of moistened non-woven sheets adapted for use on an exemplary eye compress assembly was performed. Sheets were adapted for use from a non-woven fabric material containing a mixture of pulp and polymers. Specifically, this sheet was adapted for use on an eye compress assembly by cutting the sheet to desired dimensions, and then adding water and various chemicals (considered safe for contact with the eyes and eyelid skin) to achieve a desired level of moisture. Testing showed unexpected advantages over previous materials known in the art. For example, it was found in experimentation that water-absorbent non-woven fabric sheets actually proved particularly advantageous in use with a thermally-adjustable eye compress system as described herein. The moisture was not found to serve as a thermal barrier. When moisture was added to a dry nonwoven sheet, the sheet more readily absorbed the thermal effect from the gel pack and transmitted that effect to the user. In effect, the moisture was seen to serve as a conductor of heat rather than as an insulator.

In the particular application of microwave activation for heat therapy, the moisture-containing sheets may be preferred as it was found that moistened sheets improve the even distribution of heat throughout the microwaved gel pack. Without wishing to be bound by theory, it is believed that the moist sheets may act more homogeneously in relation to microwave irradiation and, as the sheet heats up, it may pass this homogenous heating to the gel pack. Such a characteristic is unexpected since the sheet has a lower water content than the gel pack and would not be expected to influence the gel pack heating.

Paper towels are often recommended for use with gel pack systems for ocular compress therapy. Paper towels have the convenience of being widely available and, for the most part, being free of additives or chemicals that, if kept in prolonged contact in a heated and wet condition on the eyelids, might readily gain entry onto the eye surface. However, they were found to have several unexpected drawbacks when tested experimentally in comparison with non-woven fabric sheets, and particularly when tested for performance as moistened sheets.

Initially, the only drawback expected from paper towel sheets was that they would be significantly rougher in texture compared to non-woven fabrics. Dry paper towels typically have rough or “pebbled” surfaces, which may help to serve their function as drying and clean-up agents, and may make these towels more resistant to tearing during routine use. It was thought, however, that the water-absorbing characteristics of paper towels might outweigh the drawbacks of roughness, because the moisture-absorbing and moisture-retaining quality of paper towel sheets would be desirable in the field of wet compress therapy.

To gauge the water-absorbing properties of paper towels, paper towels were taken from a roll of paper towels, which were advertised as having an “outstanding absorbency” and “cloth-like durability.” These were compared to non-woven sheets made of a combination of pulp and polymers, as described above.

It was found that, as expected, the pebbly surface texture of the paper towel sheet was noticeably rougher and less pleasing to the touch than the smooth and soft surface of the non-woven fibrous sheet. However, the non-woven fabric sheet was found also to have an unexpected “springiness,” despite its relatively thin profile, that the paper towel did not have. This was seen as a positive aspect of the non-woven material because of the desirability of a cushioning effect to be provided by the sheet when interposed between the user's face and a gel pack.

In order to simulate preparation of the sheets into a pre-moistened state, both a paper towel sheet and a non-woven sheet were immersed in a shallow water bath for 10 minutes and then removed. Unexpectedly, it was discovered that the 2-ply structure of the paper towel sheet had come apart, separating into two single-ply sheets as it was being lifted from the water bath. Upon further testing, it was found that separation of the two plies of the paper towel sheets occurred after as little as 15 seconds of immersion in the shallow water bath. This was an unexpected finding because paper towels are typically made to be strong and sturdy under cleaning conditions in which they are expected to get wet. Because this discovery suggested that long-term storage of a pre-moistened paper towel may need to be performed using a single-ply paper towel, the paper towels were separated into single ply. While both single- and double-ply paper towels were tested experimentally, double-ply paper towels may not maintain integrity under packaging, shipping, and conditions of use while in the pre-moistened state based on the above-described results.

The absorption of water was tested in sheets made of three different materials: non-woven fabric, one-ply paper towel, and two-ply paper towel. Unexpectedly, the non-woven fabric exhibited the greatest water-absorption effects, showing that it could retain over five times the amount of moisture as a single-ply paper towel sheet and around 60% more water than a two-ply paper towel sheet. This was an unexpected result because the paper towel sheets were marketed, as indicated above, as being especially water-absorbent and good for cleaning up spills, whereas non-woven sheets used for cleaning are marketed primarily in relation to dirt, rather than liquid, cleanup.

The pre-moistened sheets' resistance to tearing was also tested by repeated attachment of the sheets to a support structure (i.e. repeated buttoning of a sheet to a support structure). It was found that the wet single-ply paper towel tore easily and early in this test. This was an unexpected result because the paper towels were advertised as having “cloth-like durability” and were expected to be at least as durable than the non-woven sheets, which are typically marketed for one-time use rather than for durability.

The drying time of the sheets was also compared under both benchtop conditions and under actual use with a human subject. It was discovered in various tests that a given quantity of water was retained by the non-woven sheet significantly better than by a two-ply paper towel, and even more significantly better than by a one-ply paper towel. This markedly greater retention time of moisture by a non-woven sheet was unexpected because it would be expected that two cleaning sheets of approximately the same size and weight would exhibit approximately similar characteristics, including drying time.

Thus, under testing conditions, non-woven sheets appeared to exhibit unexpectedly superior characteristics to paper towel sheets in the areas of integrity while moistened; moisture absorbence; resilience in use; and moisture retention. These are all preferred characteristics of a moistened sheet to be used with a thermally-adjustable compress, in which the compress should maintain its therapeutic value prior to use as well as during use despite repeated heatings and coolings and should also preferably be able to sustain manipulations such as re-positioning and re-buttonings to a support structure. For example, during intensive hot compress therapy, repeated 3- to 5-minute moist thermal applications over a 15 or 20 minute period are quite common, and a sheet that exhibits sufficiently necessary qualities of moisture absorption; moisture retention; and integrity are preferred to aid in providing sufficient therapy to the user.

A sheet material is not a woven cloth material, such as a terrycloth material. In daily use, knitted and woven materials would tend to present an increased risk of infection relative to non-woven fabric sheets. Knitted and woven materials are generally considered to be items of long-term and repeated use, in contrast to thin sheets or wipes made of non-woven fibrous layers, which are generally considered to be disposable after a single use or very few uses. In part, this perceived difference is due to the greater cost per unit of knitted or woven as compared to non-woven fabrics, and in part due to the fact that knitted and woven materials are likely to be sturdier and more durable. If a cloth material, such as the material used in terrycloth towels, were to be used as a sheet, it might tend to encourage long-term repeated use by the user. If regular laundering were not carried out on such sheets, the risk of buildup of dirt, bacteria, fungi, protozoa, and other materials or microorganisms could be injurious to the user, especially when users are using such sheets in a postoperative period.

The thermal barrier presented by woven or knitted cloth sheets is generally greater than the thermal barrier presented by non-woven fabric sheets. Terrycloth is an item that is typically used for compress therapy in general, including for eye compress therapy. In experimentation, a section of terrycloth was prepared for use with an exemplary eye compress assembly by cutting the terrycloth to the same size and shape as one embodiment of the facial sheet. This terrycloth sheet was then applied to a microwave-heated gel pack under various conditions. It was unexpectedly found that, despite common recommendations that users apply a cloth material over a heated gel pack, the terrycloth sheet significantly reduced the effectiveness of the microwave-prepared gel pack by blocking the thermal effect that the heated gel pack might otherwise transmit to the user's skin. A similar effect was found when the terrycloth sheet was applied to a gel pack heated in a hot water bath. This blockage occurred whether the terrycloth was wet or dry.

In controlled experiments using an exemplary device designed for ocular compress therapy, the compress device fitted with moistened nonwoven fabric sheets gave extremely satisfactory results compared to the washcloth method. The exemplary device achieved its thermal goal more quickly and accurately, and sustained the temperature at a given temperature much longer than using a thermally-adjusted washcloth. With a thermally-adjusted washcloth, the user typically has to repeatedly interrupt the treatment, removing the compress and holding it under running water or a water bath in order to reset the temperature. In contrast, there are much fewer (if any) interruptions during treatment with a gel pack and a moistened non-woven fabric sheet, owing to the sustained heat of the gel pack and the conductivity of that heat through the sheet. Because of the uninterrupted nature of the therapy, users may achieve their desired therapeutic benefit in a shorter amount of time than with the washcloth method. Thus, while the typically prescribed treatment for hot compress ocular therapy is 3 to 5 minutes based on the washcloth method, such a duration may prove not be necessary when a heated gel pack is used with a moistened non-woven fabric sheet.

During thermal compress therapy to a sensitive anatomic area such as the periocular and perioribital regions, it may be desirable to selectively focus the thermal effect on one body region (the “thermal target region”) while sparing or diminishing a thermal effect on a body region that is immediately adjacent to the thermal target region. For example, it may be desirable to selectively focus a thermal effect on the periocular region, while sparing a thermal effect on the periorbital region or the nasal bridge. It may, conversely, be desirable to focus a thermal effect on the periorbital region while sparing a thermal effect on the periocular region.

In the art of thermal compress therapy using gel packs, such selective thermal application and thermal sparing of adjacent tissues is generally achieved through the shape of the gel pack itself. In other words, gel packs in the art are generally shaped and sized in order to provide a surface area that roughly corresponds to the surface area of the selected anatomic region, so that their thermal effect is transmitted over the whole of the area of contact between the surface area of the thermal compress and the user's anatomy, thus sparing a thermal effect to any areas outside that area of contact. For example, certain compresses are shaped and sized to selectively apply a thermal effect only to the periocular regions, but not to the periorbital regions, and such compresses are therefore shaped and sized so that they only cover the periocular regions and not the periorbital regions.

In one embodiment of the current invention, an eye mask shaped thermal gel pack is designed to cover a relatively large surface area of the face (including both the periocular and periorbital regions), even under circumstances in which the thermal target region (for example, the eyelids) is considerably smaller than the entire area of coverage of the gel pack. One way to create a thermal barrier in one anatomical location and a thermal transmission area in another location under a single area of the gel pack, is through the selective use of dry and moist areas on a sheet or layers of sheets that are interposed between the gel pack and the user's skin.

It has been found that when moisture was added to a water-absorbent removable facial sheet that was placed between a heated or cooled gel pack and the user's skin, the thermal effects of the gel pack were greatly increased (that is, the water served as a source of thermal conductivity from the gel pack to the skin). This finding was unexpected, because it was directly contradictory to the statement by Helming (described above) that water tends to serve as an insulator and to reduce the effectiveness of thermal therapy.

It has also been found that while the use of moist facial sheets or layers of moist facial sheets will tend to readily conduct the thermal effect of the gel pack, the use of dry facial sheets or layers of dry facial sheets will tend to resist the thermal effect of the gel pack, and thereby shield certain areas from the thermal effect.

Experiments were performed to show that selective regions of the areas underlying the gel pack could be targeted for thermal therapy through modifications of the facial sheets and layers of facial sheets interposed between the gel pack and the skin.

As an example, an eye mask shaped gel pack and an eye mask shaped moistened sheet were microwave-heated and used to simulate hot compress therapy in an experimental setting, with the moistened sheet lying against the user's periocular and periorbital areas and with the gel pack resting outside the sheet. For purposes of convenience, this will be called the “basic moist heat system.”

The basic moist heat system was then modified in various ways to selectively target heat therapy to the periocular regions. For example, a dry non-woven sheet was created with the same perimetric size and dimensions as the moistened sheet, but with eye-shaped apertures (horizontal ovals) created in the surface of this sheet. Of course it is possible that other materials for the sheet could also be used. Waterproof envelopes were also created in order to contain the dry sheet and keep it from becoming wet. These envelopes were prepared from heat-conductive but waterproof films; one made from poly(vinyl chloride), and the other made from a polyethylene-containing film. The dry sheet was applied to the basic moist heat system in two methods: first, directly; and second, contained within one of the waterproof envelopes.

Starting with the basic moist heat arrangement (that is, with the moist sheet lying sandwiched between the user's face and the heated gel pack), the dry sheet with eye-shaped apertures was interposed between the moist sheet and the user's face. The material of the dry sheet substantially reduced heat transmission to the face in the periorbital area (areas that were covered with the dry sheet), but allowed full application of such heat in the periocular regions (areas that were exposed to the wet sheet through the eye-shaped apertures cut into the dry sheet).

In a separate experiment, again starting with the basic moist heat arrangement, the dry sheet with eye-shaped apertures was now interposed between the gel pack and the moistened sheet. A similar effect as above, sparing the thermal effect on the periorbital but achieving it on the periocular regions, was obtained.

The effects of selective thermal application were achieved adequately both with and without the dry sheet being enclosed within waterproof envelopes.

It was unexpected that a dry sheet disposed directly on a moistened non-woven sheet served as a thermal protective barrier since it would be expected that the moisture from the moistened sheet would seep through to the dry sheet. Without wishing to be bound by theory, it may be that because of the exceptional ability of non-woven materials to hold on to water and to resist the spread of such water by capillary action, that moisture is not actively transmitted to the dry sheet and that it can stay dry for a long enough period to fulfill its thermal barrier function.

The present invention also provides a similar method in which the dry sheet is prepared with slits, sized and spaced to allow selective treatment of the eyelid margins only, which would be effective in targeting thermal therapy for the eyelid margins. Similarly, the present invention provides a method in which sections of a dry sheet (possibly a waterproof sheet, or possibly a dry sheet encased in a waterproof envelope) is prepared so that a portion of dry sheet only covers the periocular regions, but has no barrier or cover over the periorbital regions, such that this specially-shaped sheet would produce selective heating around the eyes but not on the eyes or eyelids themselves. In addition, the present invention provides a single sheet, which is pre-treated so that it has both wettable and non-wettable areas that achieves the same effects as the two-sheet method described above. In other words, the wettable area of such a sheet selectively transmits thermal therapy to the target tissues underlying the wetted areas, and has a thermal barrier effect over the tissues underlying the non-wettable areas.

The selective application of heat could also be applied such that the portion of the facial sheet that covers the nasal region is kept dry, to reduce the amount of heat transmitted to this particular area, for the comfort of the patient.

Although the above embodiments for achieving selective thermal barrier effects involve using removable sheets or layers of sheets, such selective barriers (dry and/or wet sheets) can be permanently applied to a gel pack or to an external support structure (such as the external frame described in more detail below). For example, a portion of waterproof non-woven material or an otherwise dry sheet, shaped to cover the back side of a gel pack except for horizontal oval-shaped regions where the user's eyes would be expected to sit, could be used to provide a more permanent thermal effect that would target therapy for the periocular regions. Of course, other configurations of the dry sheet(s) could also be used to target different areas for therapy.

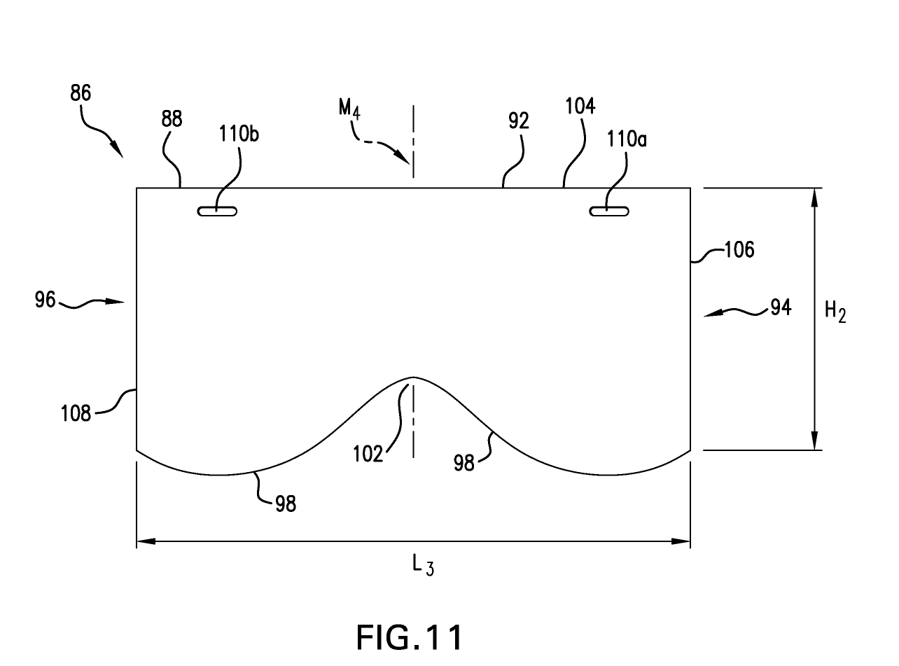

The present invention also provides an eye compress kit that includes a gel pack and a plurality of sheets, each of which can be positioned between the gel pack and the body region of the user. A kit can also include a dispenser that can hold the plurality of sheets. For example, referring to FIG. 12, in an embodiment, a dispenser 112 comprises an inner receptacle sized to hold a plurality of stacked facial sheets. In the embodiment, the dispenser is in the form of a rigid substantially rectangular box having a bottom side (not shown), a top side 116, and lateral sides 118 (only two lateral side shown) but other configurations are also possible. In this embodiment, top side 116 comprises a slit 122 sized to allow at least one of the plurality of facial sheets to be removed from receptacle 114. Slit 122 can be covered by a removable adhesive covering applied against the slit to close the slit after a facial sheet(s) has been removed and to therefore protect the facial sheets from drying out. In the embodiment shown in FIG. 12, the top side of the lid comprises a movable lid 114 that can, but does not have to be, pivotably mounted to at least one of the lateral sides or to the top side of the dispenser. In certain embodiments, the lid is removable. In still other embodiments, there is not a separate lid that moves on a pivot that is connected to the top or lateral side of the dispenser, but rather the entire top side 116 of the dispenser serves as lid and is capable of being opened and closed either via a hinged element or not.

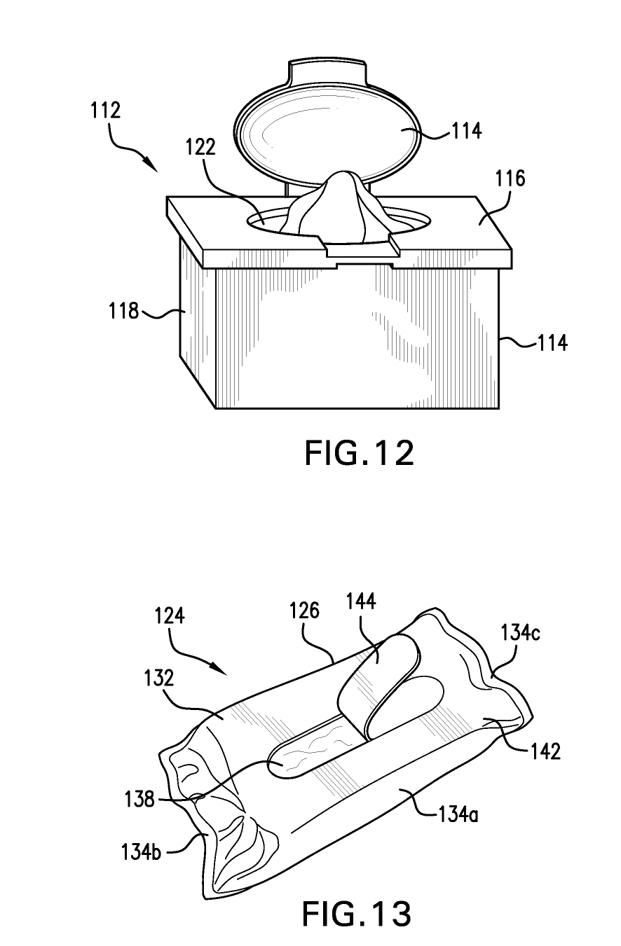

Referring to FIG. 13, in other embodiments, a dispenser 124 is in the form of a flexible wrapping 126. Wrapping 126 has a bottom side (not shown), a top side 132, and lateral sides 134a, 134b, 134c, and 134d (not shown) that define an inner receptacle. Top side 132 comprises a slit 138 sized to allow at least one of the plurality of facial sheets to be removed from the receptacle. Slit 138 can be covered by a removable adhesive covering 144 applied against slit 138 to close slit 138 after a facial sheet(s) has been removed and to therefore protect the facial sheets from drying out.

In certain embodiments, a compress assembly and system further comprise an external frame attachable to or otherwise positionable against the front side (outwardly facing side) of a gel pack to compress the gel pack against the user's anatomy. The external frame can be actively attached to the gel pack via mechanical means such as fasteners (described in detail below) to form a compress assembly or can be passively positioned against the gel pack without any mechanical means physically attaching the external frame to the gel pack to form a compress system. In the latter embodiment, in use, the external frame can apply backward pressure against the gel pack and sheet via a strap that is attached to the external frame and secured around the user's head in order to maintain the relative positions of the gel pack and sheet against the user's anatomy.

In certain embodiments, the external frame is actively attached to a gel pack and a sheet to both compress the gel pack and sheet against the user's anatomy and to vertically support at least part of the gravitational weight of the gel pack and sheet when the compress assembly is in use (i.e. in an applied position), in order to maintain the gel pack in a relatively vertical and planar (flat) configuration against the body part, rather than having the gel pack sag down under its own weight into a relatively non-planar configuration. In particular, an external frame is fabricated from a material stiff enough to support the weight of the gel pack such that the frame does not buckle when the gel pack is attached to or positioned on the external frame when the external frame is in a vertical orientation and the gel pack is secured against the body region of the user. An external frame can also help position the gel pack and the sheet properly in relation to one another and in relation to the particular anatomic location desired by the user in order to best direct the therapeutic aspects of compress therapy to the desired areas. Specifically, when used, an external frame stabilizes the gel pack and the sheet as a unit, making them easier to handle during preparation so that they do not slip in position in relation to one another when the unit is held and then applied over the user's body portion (such as the head in the case of an eye compress assembly).

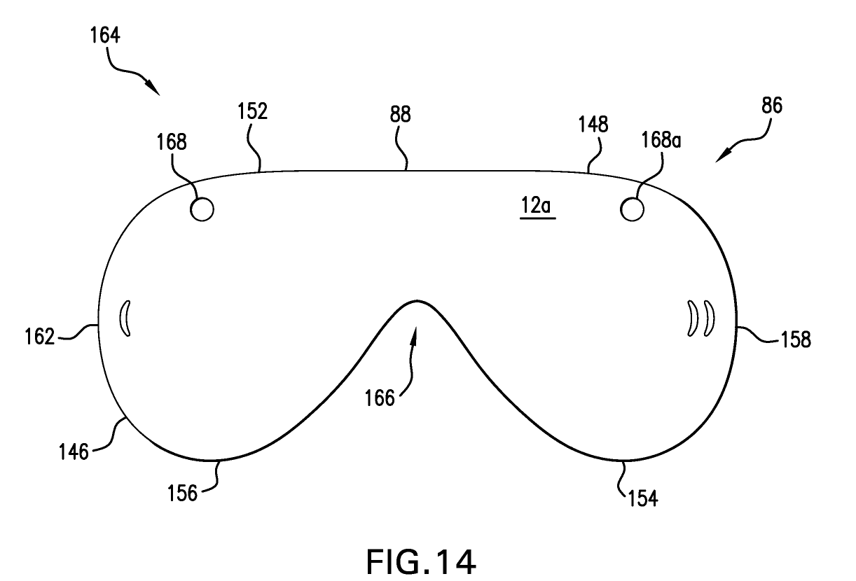

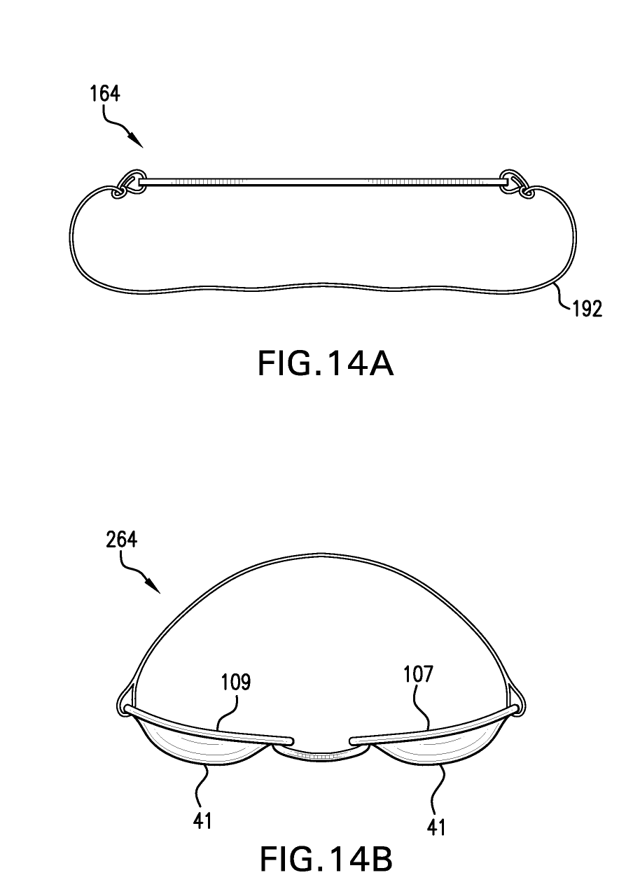

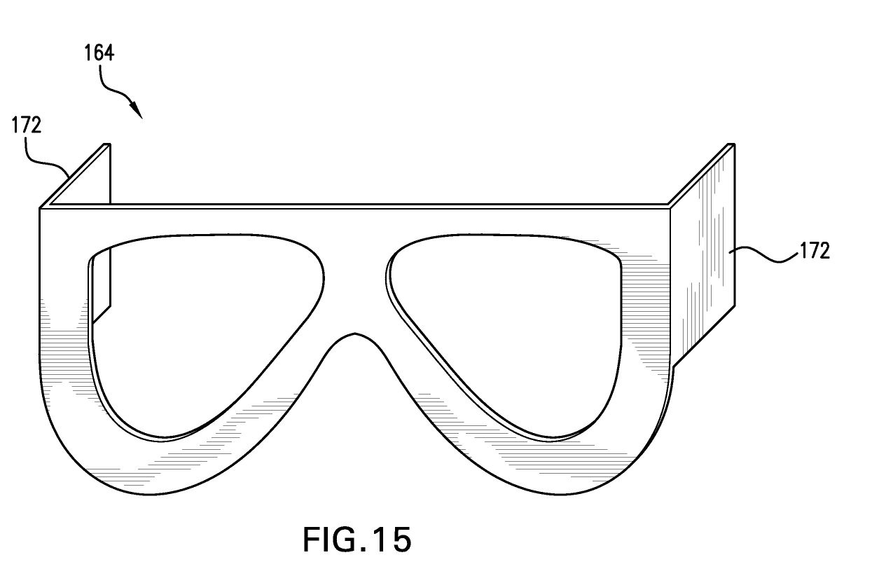

As seen in FIG. 14, in an embodiment, external frame 164 comprises a frame body 146 having a top left portion 148, a top right portion 152, a bottom left portion 154, a bottom right portion 156, a left side portion 158 and a right side portion 162. In the embodiment shown in FIG. 14, all the aforementioned portions of the frame body are curved but some or all of the portions can have straight edges as do the gel pack and facial sheet of FIGS. 2 and 11, respectively. In the embodiment shown in FIG. 14, the right and left side portions transition into a bottom portion shaped like a bell curve which essentially creates a curved cut out 166 to accept the nasal wings of the user. Alternatively, the peak of the cut out could be angled instead of curved (similar to the notch 225 of gel pack 10 shown in FIG. 2). Preferably, an external frame mimics the outline of the gel pack that is used with the external frame as part of the eye compress assembly or system. For example, if the gel pack is configured as shown in FIG. 5, external frame 164 can also be similarly figured, as shown in FIG. 15 with lateral wings 172 such that, in use, external frame 164 applies compression to the temples. Of course, an external frame could be configured to have lateral wings that cover the temples of the head during use irrespective of the configuration of the gel pack. In any event, preferably, an external frame is sized and shaped such that the gel pack extends beyond the edges of the external frame on all sides so that the user's face is contacted in all applied areas by the gel pack rather than directly by the external frame. Similarly, referring to FIG. 16, in embodiments where a facial sheet is used, preferably the external frame 164, gel pack 20 and facial sheet 86 have similar outlines with facial sheet 86 having a greater surface area than gel pack 20 and gel pack 20 having a greater surface than the external frame 164.

In certain embodiments, the external frame assumes a generally flat or planar conformation when in a resting position. As used herein, a “resting position” refers to the position of the external frame when it is not applied against the body region of the user (i.e. an applied position) and is resting on a flat surface. This resting position of an external frame can be seen best in FIG. 14A, which is a plan view of the external frame 164 of FIG. 14. This generally flat or planar configuration is in contrast to the configuration of external frame 264 in FIG. 14B, which has a convex outer surface 41 for the section 107 that covers the right eye region and the section 109 that covers the left eye region (which is the surface that would face away from the user's face in an applied position). Thus in embodiments where the external frame has a generally planar configuration, when the eye compress assembly is applied against the eye region, the external frame has the ability to press the gel pack directly against the desired eye regions (i.e. the periocular and/or periorbital region of the face), which would not occur if the external frame had a convex configuration in a resting position.

As described above, in certain embodiments, an external frame is attached to the gel pack and the external frame is fabricated from a material stiff enough to support the weight of the gel pack such that the external frame does not buckle when the gel pack is attached to the external frame and the external frame is in a vertical orientation when the gel pack is secured against the eye region of the user. An external frame can be attached to the gel pack in any suitable way. For example, the external frame can be permanently or removably attached to the gel pack in use. Regarding the former, an external frame could be glued or heat molded onto the gel pack during manufacture. Other means of permanently attaching an external frame to the gel pack are also possible. If an external frame is permanently attached to the gel pack, the external frame is fabricated from a material that is heat and cold resistant such that the external frame can be exposed to a heat or cold source without degrading to the point of losing its intended functions.

Regarding an external frame being removably attached to the gel pack in use, the frame can accommodate at least one fastener to secure the gel pack to the external frame. For example, as illustrated in FIG. 14, the external frame can define apertures 168a and 168b in top portion 148 and 152, respectively, of frame body 146 that are configured to receive buttons, string, snaps or other fasteners to attach to the top portion of the gel pack, in which case the external frame supports substantially all of the gravitational weight of the gel pack. Although FIG. 14 illustrates two apertures, an external frame can include more than two apertures in the top portion of the frame body. An external frame could also define any suitable number of apertures in bottom right and left portions 156 and 154 respectively and/or right and left side portions 162 and 158, respectively. Alternatively, external frame 164 could define a single aperture centrally located between top right portion 152 and top left portion 148. Still alternatively, the external frame could define at least one aperture on left side portion 162 and at least one aperture on right side portion 158 of frame body 146. In such an embodiment, the frame body supports at least a portion of the gravitational weight of the gel pack (but not as much as would be supported if the gel pack were attached to the top portion of the frame body). However, the gravitational weight that is supported in such an embodiment is enough to prevent the gel pack from sagging during use. The exact number and location of the apertures can vary so long as an external frame supports enough of the gravitational weight of the gel pack such that the gel pack does not sag in an applied position. Notwithstanding the exact number and location of apertures in external frame 164, the gel pack and sheet would have similar apertures, for example, as described above such as with respect to FIGS. 2 and 11.

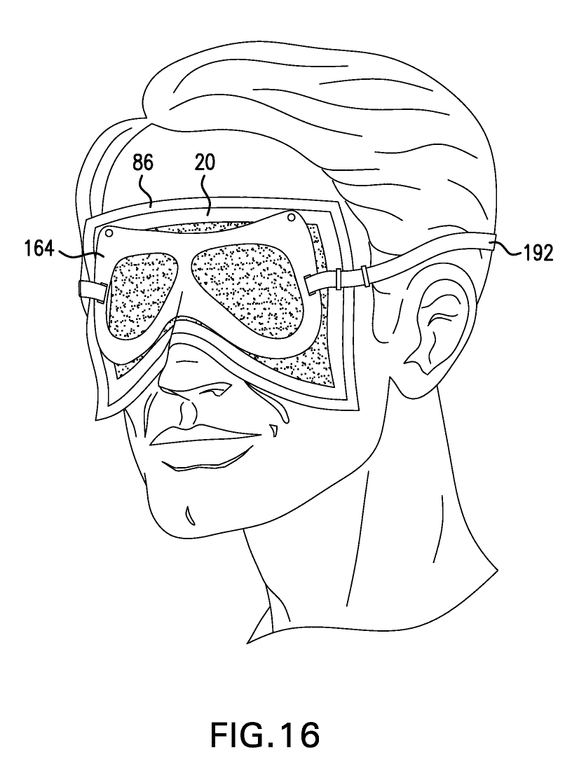

In addition to being designed to receive separate fasteners that are applied to the frame body, the frame body can contain fasteners that are already attached or attachable to the frame body. For example, the frame body can accommodate a magnetic strip to attach to a magnetic strip or metal strip disposed on a gel pack. In turn, a sheet can have a magnetic strip or metal strip to attach to the gel pack. In certain embodiments, as shown in FIG. 17, external frame 164 can have buttons or other fasteners already attached to the frame body. Such fasteners can be die-cut or molded, for example, into the frame body of the external frame. For example, FIG. 17 shows buttons 174 attached to the frame body of external frame 164. Of course the aforementioned fasteners are only exemplary and other fasteners can also be used so long as they achieve the function of attaching a gel pack and a sheet to an external frame. Further any number of fasteners can be used to secure the components of the eye compress assembly. Non-limiting examples of other types of fasteners include velcro, clips, and male/female fasteners. As such, an eye compress kit can include separate fasteners that are not integral with the external frame but rather separable in the sense that the fasteners can be separated from the external frame without disrupting the integrity of the external frame or fastener (i.e. tearing or breaking the external frame or fastener). An example of such separable fasteners include clips as shown in FIG. 10B. In preferred embodiments, the fasteners are attached or attachable to the top portion of the frame body although the fasteners can be positioned at different locations on the frame body as described above with respect to apertures defined by the frame body to receive fasteners.

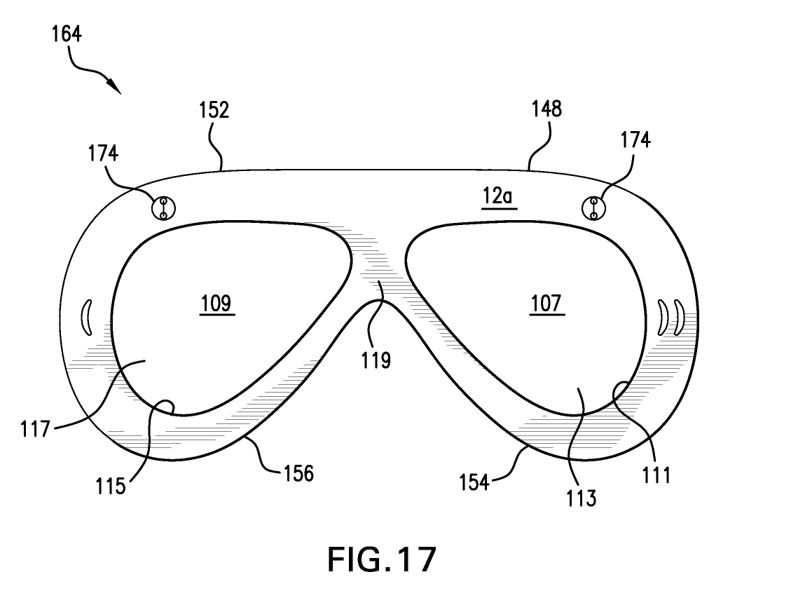

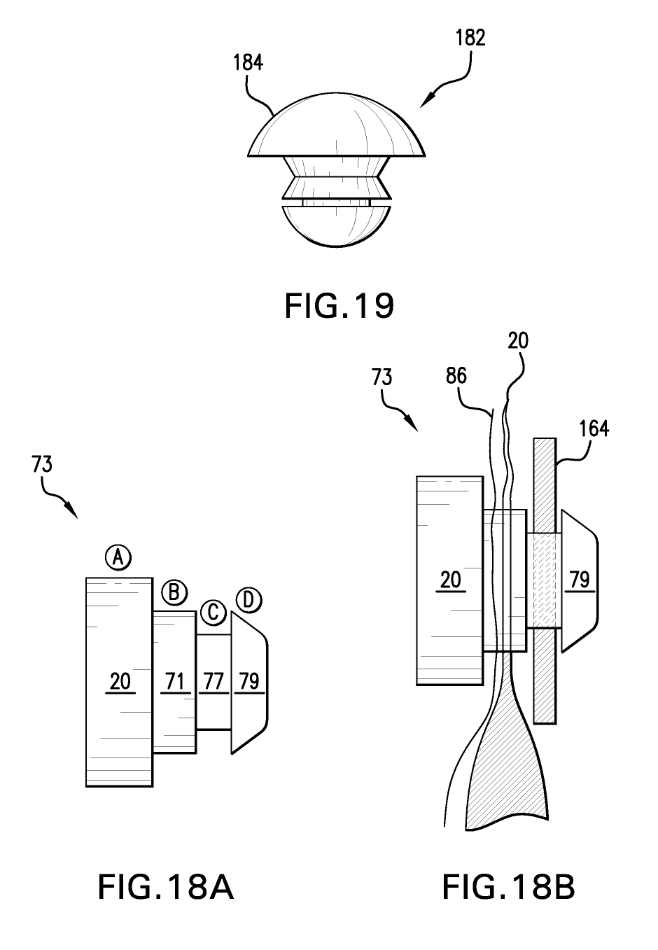

With specific reference to fasteners that are buttons, button-shaped fasteners may allow a broad range of users to intuitively understand the removable nature of the gel pack and the sheet Referring to FIGS. 18A and 18B, in certain embodiments, a fastener 73 comprises a button 20 and a stepped down support shaft 71 connected to or integral with button 20 and sized to allow room for the insertion of both the top portions of the gel pack 20 and the sheet 86 as shown in FIG. 18B. Fastener 73 further comprises a post 77 that passes through the external frame 164 as shown in FIG. 18B and a stepped up anchor 79 that is on the opposite side of the frame as button 20 and that keeps button 20 locked to the external frame 164. Such buttons could be made of a variety of materials such as plastic or rubber. Buttons made of a soft silicone material were found to impart a flexible tension that allowed them safely and snugly to grip, rather than just passively support, the wet sheets used with the device and are therefore preferred (although not required). In one embodiment, shown in FIG. 19, a button 182 is designed and configured with a domed surface 184, rather than in a standard shape, in order to improve the ease with which users insert moistened sheets onto the buttons.

As described briefly above, in embodiments where a facial sheet is desired to be actively placed between the user's eye region and the gel mask, the facial sheet can also be configured to receive fasteners. For instance, as shown in FIG. 11, the top portion of the facial sheet can define apertures 110 to accommodate the same fasteners used to secure the gel pack to the external frame.

An eye compress assembly that includes an external frame also includes a strap 192 as shown in FIG. 16. The strap can be attached to the gel pack or the external frame but in the embodiment shown in FIG. 16, strap 192 is attached to the left and right portions of the external frame 164. In this embodiment, the strap is adjustable (although it is not required to be in other embodiments) such that tightening or loosening of the strap exerts a controllable horizontal pressure on the external frame. In certain embodiments, where an external frame, a gel pack and a facial sheet are attached together via their top portions as shown in FIG. 16, this pressure is largely independent from the vertical support provided by the external frame to the gel pack via the one or more fasteners at the top portion of the eye compress assembly. As shown in FIG. 16, to achieve this effect, in certain embodiments, an external frame can include slits in the right and left side of the external frame to accommodate a strap (such accommodation being similar to that described above with respect to a gel pack). Alternatively, a strap can be attached to an external frame via other exemplary means such as by being stitched or glued onto the external frame (again, such exemplary means being similar to that described above with respect to a gel pack). Therefore, the strap can be permanently or removably attached to the external frame. The strap can be made of a variety of stretchable or non-stretchable materials that preferably will not be either adversely affected by low levels of heat or heated by low levels of microwave irradiation such as the amount required to heat the gel pack, including fabrics, plastics, woven elastics, and some pliable elastic polymers. Preferably, the strap is able to be loosened sufficiently so that the external frame can serve simply to support the gel pack and soft sheets in relation to the body part, without compressing them against the body part. In general, the description of a strap as described above with respect to a gel pack applies to a strap attached to an external frame instead.

Referring back to FIG. 17 in certain embodiments, external frame 164 further comprises a bridging portion 119 that bridges the top portion (both left and right top portions 148 and 152) and the bottom portion (both left and right bottom portions 154 and 156). In this embodiment, external frame comprises a left section 107 whose internal periphery 111 defines a left relief opening 113 and a right section 109 whose internal periphery 115 defines a right relief opening 117. The relief openings are sized to allow a user to directly manipulate the position of the gelatinous substance in the gel mask by applying topical pressure to the gelatinous substance, a feature that is useful when the overall compressive tension in the frame is kept low but the user wants to selectively increase the compress effect in certain areas. Such relief openings also reduce some of the compressive pressure that would come from increasing the backwards tension on the frame. For instance, the relief openings allow the frame to focus such pressure on the periphery of the targeted body area rather than on the center of that area. The relief openings can have any suitable shape such as tear-shaped or circular, for example.

The relief openings can be directly exposed to the atmosphere or can be covered with a thin layer of fabric, plastic, foil, or other material which would cover the gel pack underlying the openings but would be flexible enough to allow the user easily to manipulate the gel. Certain materials could be selected to insulate the gel pack by reducing the amount of convective heat exchange with the surrounding air.

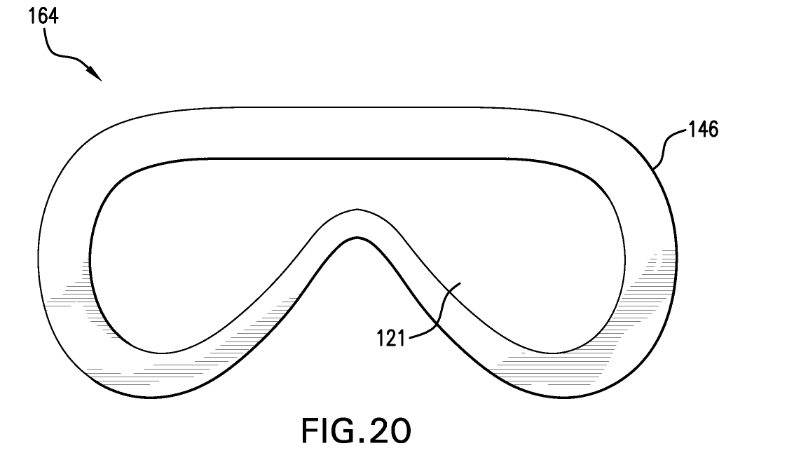

Referring to FIG. 20, in certain embodiments, the internal periphery of frame body 146 defines a single relief opening 121 which serves the same purposes as the dual relief openings described above with respect to FIG. 17.

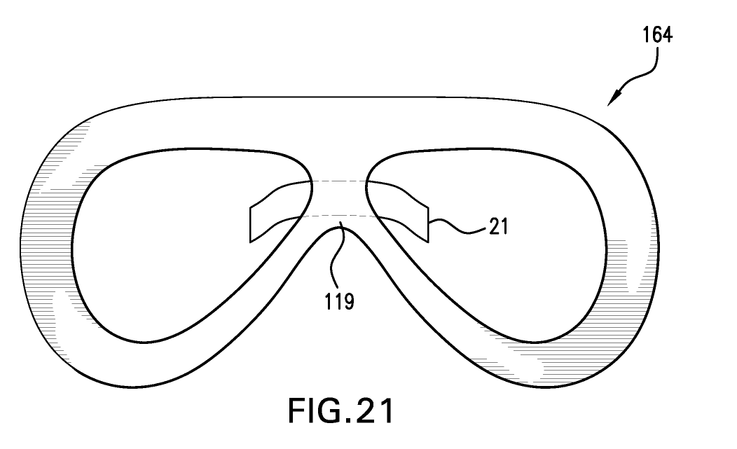

In reference to FIG. 21, in certain embodiments, an eye compress assembly further comprises a gel impressor 21, which is a flexible bendable strip that can be placed in contact with the back surface of bridging portion 119 (i.e. the surface that would be in contact with the gel pack). Gel impressor 21 can provide a hands-free option for creating selected indentation in certain areas of the gelatinous substance of the gel pack. Some users who use towels for wet compress therapy have noted a desire to keep mild pressure in the specific area of the nasal corners of each eye (the areas overlying the nasal canthi) in order to provide extra comfort and relief of symptoms. Gel impressor 21 provides a way for users to apply such directed therapy at these locations. In one embodiment, the gel impressor is made of a flexible and bendable material that retains the shape to which it is bent. Soft metals such as aluminum are one example.

An exemplary method of using a gel impressor will now be described. An external frame attached to a gel pack is provided. An experimental gel impressor is made from consumer-grade aluminum foil which is folded on itself several times to create a multi-ply sheet 2½″ wide by ½″ high. This impressor is placed on the back surface of the bridging portion of the external frame in a horizontal configuration (such that the width of the impressor is in the horizontal plane). The user is then free to squeeze the two ends of the impressor back toward the nasal canthi in a very natural manner (similar to pinching the bridge of the nose between the thumb and forefinger). This achieves a desired targeted effect of having the ends of the impressor continue to press the gelatinous substance of the gel pack in toward the nasal canthii, and the user can thereupon continue to experience the therapeutic benefit of the eye compress assembly in a hands-free manner. While the illustrated gel impressor is positioned to apply pressure over the nasal canthal regions, an impressor could be repositioned and applied to other anatomic regions as well.

Referring back to an external frame and specifically to materials that can be used to manufacture an external frame, when the external frame is used to support at least a portion of the gravitational weight of the gel pack, the external frame material should be stiff but bendable enough to serve its intended function. That is, when an external frame, sized and shaped for anatomic use in a particular area, including optional relief openings, is placed vertically upright and a gel pack that is also designed for such anatomic use is attached to the external frame, the external frame should be stiff enough to resist buckling or bending, thereby supporting the gel pack's weight and maintaining its shape. However, when the external frame is bent over a body part, such as when the external frame is bent to drape over the nasal bridge, the external frame preferably exhibits flexibility so as to conform to some degree with the external contour of the body part (such as the face), rather than remaining in a stiff, flat, and unbent configuration. This flexibility should be preferably of a sufficient degree that, when the frame is subjected to forces provided by the materials mentioned above as possible contents of the strap, the frame will bend over the body part and thereby press against the underlying gel pack along the full extent (height and length) of the underlying gel pack. A flexible material may also allow the frame to be folded easily in half, down its central midline, a feature which would allow convenient insertion into a case that would be sized and shaped for the express purpose of containing the external support for travel and/or storage. A microwavable material is preferred, although it is possible to use a non-microwavable material. A waterproof material is preferred because of the expected use of wet sheets as part of compress therapy, although the wet sheets would not tend to come into direct contact with the external frame during routine use.

With respect to specific materials from which the external frame can be fabricated, any one of a variety of plastics may be suitable including, but not limited to, polymers such as polyethylene, polypropylene, polycarbonate, polymethyl methacrylate, polyethylene terephthalate, co-polymers thereof, and combinations thereof. Polypropylene (as well as other plastics) are easily dyed to different colors, a factor that could allow easy and unique identification of external support among different users in a household. Additional materials that may be used include stiffened foams, cardboard or similar paper materials, self-welted and/or stiffened fabrics, and the like. If permanently attached to the gel pack or in other circumstances where the external frame is heated with the gel pack, the material of the external frame preferably shows no significant degradation under repeated exposure to microwave radiation. The definition of “significant degradation” is the same in this context as described above with respect to a gel pack. In experimentation, a 0.030″ (30 gauge) sheet of polypropylene, die-cut to the design shown in FIG. 17 was found to be lightweight and comfortable in use, resistant to sagging or stretching, readily bendable over the nasal bridge, and resistant to multiple (greater than 50) exposures to heat and to microwave irradiation.

In certain embodiments, an external frame is covered or layered with a fabric or other soft or flexible material to provide a softer external surface, in order to improve user comfort when handling the external frame.

In general (as described above) the dimensions, shape and peripheral contour of the external frame are preferably designed to complement the dimensions, shape, and contour of the gel pack. This will provide a source of variable compressive pressure that will push the gel pack more firmly against the body if so desired by the user. For the user's greater comfort, however, the frame can be cushioned by gel in any area where it might otherwise press directly against the user's skin. To achieve this outcome, in certain embodiments, the frame edges are generally designed with a smaller perimeter than the gel pack edges. Such design may be altered depending on the particular anatomic location to be treated. In the particular embodiment of an eye compress device shown for illustrative purposes, the top portion of the external frame, in use, can sit high enough above the eyes to allow for secure placement of fasteners that secure the top edge of the gel pack. The cut out in the bottom portion of the external frame is preferably raised to provide clearance above the bridge of the nose, so that the frame does not exert pressure on the nasal bridge even when the frame is adjusted to transmit a greater compressive force against the gel pack.

The external frame is preferentially designed to support and maintain the soft gel pack and sheet in position against the body, without the need to forcibly strap or compress these elements into position in order to keep them in place. The adjustment of the intensity of compression of the gel pack is preferentially achieved through means (such as via a strap) that are largely independent from the support functions of the external frame.

The below exemplary description of an exemplary eye compress assembly will illustrate these principles. First, the support action of the external frame will be explained. When the user of an eye compress assembly, such as that shown in FIG. 16, for example, is in an upright position, the top portion of gel pack 20 and the top portion of sheet 86 are affixed to the top portion of external frame 164. The frame itself is made of a material that does not sag when supporting the weight of the gel, so that the top portion of frame 164 maintains the top portion of gel pack 20 at a specified height in relation to the anatomy. The bottom portion of frame 164 may be designed so that it does not touch the user's skin directly, but is instead cushioned by the lower border of the bottom portion of the gel pack, while the top portion of external frame 164 maintains the support of the top portion of gel pack 20.

Next, the compressive action of the frame will be explained. When the user of the illustrated eye compress assembly is in an upright position and the strap is placed around the head with minimal tension, the strap may be loose enough so that no compressive force is transmitted to the user's face. In this case, the bottom portion of the external frame rests upon the upper portion of the cheek, and the upper portion of the frame is tilted away from the eyes, so that the gel pack and the sheet remain in front of the eyes but without necessarily coming into direct contact with the eyelids or periorbita. When the user desires to increase the compressive intensity of the compress assembly, the user adjusts the strap in order to increase tension in the strap, possibly by using a buckle or other type of strap-adjusting mechanism. Under tension, the ends of the strap pull back against both the left and right side of the external frame creating a backwards tension on the frame that is transmitted onto gel pack and sheet, thus pressing these elements inwardly against the user's face.

In this exemplary description, the fasteners that keep the gel pack in a vertical orientation are kept in one area of the frame (in the eye compress assembly example, this is at the top portion of the frame), whereas the strap allowing adjustable transmission of tension, and the generation of a compressive force, are kept at another area of the frame (in this example, at both side edges of the external frame). In this exemplary description, the support for the proper positioning of the gel pack and the sheet in relation to the eyes comes from the vertical transmission of their weight onto the relatively stiff frame element. In contrast, the compressive effect that the external frame exerts against the gel pack and sheet comes from the horizontal transmission of tension, which is effected by the surface area of the frame.

Because excessive tension in the strap is not needed to keep the gel pack in position against the body during normal use in this exemplary description, excessive pressure is not needed when applying compress therapy to certain sensitive body tissues. For example, in ocular compress therapy, users with sensitive eyes may relax the strap tension considerably, achieving a very low amount of inward pressure. Indeed, in use it was found that the tension could be adjusted to such a low level that the gel pack and sheet could, when their temperature was at an uncomfortable extreme, be held a small distance away from the eyes, rather than directly touching the eyes. This allowed the transmission of a thermal effect because of the near proximity of the gel pack and sheet. In the particular instance of hot compress therapy using a moistened sheet, the effect of having an overheated wet sheet remain near, but not touching, the eyelids, was an unexpectedly therapeutic application of steam to the eyelids. When the device cooled to a more comfortable temperature, tension in the strap could be increased, and contact between the device and the user's skin could be achieved, resulting in additional therapeutic warming.

In embodiments including relief openings in an external frame, when the compressive effect of the frame is increased, the relief openings can reduce the direct transmission of compressive force directly onto the sensitive body tissues underlying these relief openings. In the eye compress embodiment, for example, when the compressive force of the frame is increased, pressure is transmitted preferentially to the peripheral or periorbital areas rather than directly onto the eyes themselves. The selective application of peripheral pressure may have a secondary benefit, by squeezing gel centrally into the anatomically-shaped relief openings and thus placing a larger volume of thermally-adjusted gel directly over the eyes or periocular areas, thus enabling a prolonged thermal effect.

There are many methods for preparing a compress assembly for use. For example, a compress assembly can be heated by exposing the assembly to a heat source such as an oven, including a microwave oven or a hot/warm water source, such as a water bath. A compress assembly can be cooled, for example, by exposing the assembly to a cold source such as a freezer or refrigerator or an ice/cold water bath.

Exemplary methods of preparing a compress assembly for use will now be described with respect to an eye compress assembly. During use, compress therapy can take place with the gel pack at room temperature, heated, or cooled. In this example, a gel pack containing 2.5 ounces of gel at room temperature (around 72° F.) was used for testing purposes. Cooling the gel pack by placing the gel pack in a conventional household freezer for as little as 2 minutes resulted in adequate cooling for up to 5 minutes of gentle cold compress therapy. Longer freezing times produced a more lasting cold effect. This same gel pack was then subjected to microwave irradiation. Activation in a 1,000-watt microwave oven set on “high” for 20 to 30 seconds produced heating of the gel pack to a maximum temperature of 125 to 165° F. Based on the thermal decay characteristics, it appeared that less than a 30 second activation in the microwave produced sufficient heating for up to a 5 minute application of hot compress therapy. During testing, if a warmed pack had diminished in temperature to an undesirably lukewarm temperature, a 10-second microwave re-activation of the device was sufficient to reheat the gel pack for continued use as a hot compress.

Another exemplary method of preparing a compress assembly for use will now be described. When the user needs to apply a relatively light-weight gel pack to a body part for hot compress therapy and does not have access to a microwave oven, alternate heating methods may be useful, and the present invention provides embodiments for preparing a compress assembly with such alternate methods in mind. For example, a light-weight (e.g., 2.5 ounce) mask-shaped gel pack can be easily placed in a 12-ounce cup to which 8 ounces of boiling-hot water can then be added. In testing, this procedure heated the gel pack to an adequate temperature in less than 60 seconds. This alternative heating method may be important for users who do not have access to a microwave or a pot of hot water at the time that they desire treatment (for example, while traveling or at work), but who could easily obtain a cup of freshly-boiled water in such circumstances. A similar scenario, using a cup of ice water, would apply to travelers in need of cold compresses.

While microwave activation of an eye mask shaped gel pack is convenient for users, the nature of microwave activation and the shape of the gel pack can create potential issues with irregular heating. For example, microwave wavelengths are 12.25 cm, and objects that are longer than 12.25 cm may tend to get “hot spots” when heated in conventional microwave ovens, even when microwaves are fitted with turntables and internal “mixers” that help to distribute the microwaves and prevent standing waves. Experimentation with microwave activation of the preferred eye mask shaped gel pack confirmed that random and unpredictable heating patterns were often produced in the gel pack.

Having “hot spots” and uneven heat distribution within a hot compress that is intended for use on a particular anatomic area may be problematic. If one area of the hot compress is much hotter than other areas, such that it is too hot to apply comfortably and safely to the skin as a whole, the user may have to wait until the hottest portion of the compress cools to an acceptable temperature. By waiting until such time is reached, the remaining mass of the gel pack may cool to a temperature that is no longer warm enough to meet the user's needs.

The problem of uneven distribution of microwave heating within a hot compress has not previously been addressed in the art. There may be several reasons for this. Larger gel packs intended for nonspecific use on a variety of body parts, when microwaved, may not display the extreme temperature differences found within a relatively low-volume gel pack intended for use on a particular and sensitive body part such as the ocular and periorbital regions. In addition, certain microwave-activated heating devices known in the art of hot compress therapy to the eyes, such as placing rice or beans into a clean athletic sock, activating the device in a microwave, and then applying the heated device to one eye at a time, involves the use of a device (a handful of rice or beans that are accumulated at the bottom of a clean athletic sock) that is somewhat spheroid in configuration and does not exceed 12.25 cm in any dimension, and would thus not be affected by hot spots that affect the relatively long and flat eye mask shaped gel pack as illustrated in the preferred embodiment shown earlier.

In order to address the problem of uneven heat distribution, at least three successful methods were discovered. These were the water bath immersion method; the water-absorbent thermal regulator method; and the rapid-mixing method.

Experimentation found that immersing the gel pack in a shallow water bath prior to microwave activation, such that no portion of the gel pack was exposed to air, and exposing the water bath containing the gel pack to microwave activation, allowed even heating of the gel pack, without hot spots. Upon observation, it appeared that the water itself did not heat up quickly or high enough to be the source of the heating within the gel pack. In other words, the effect produced was not that of a hot water bath into which a gel pack is placed, such that the hot water directly conducts heat to the gel pack and serves as its source of heat. Instead, it was clear that the gel within the gel pack was heated directly by microwave activation. However, the presence of the water bath somehow modified the activation of the gel pack sufficiently enough to reduce or nearly eliminate hot spots in the gel pack itself. This water bath can therefore be considered a “thermal regulator” for gel pack activation.

The practice of using a non-heated volume of water within which the gel pack is placed, so that both gel pack and the volume of water are microwave-activated together, for the purpose of producing an even heating of the gel pack, is not known.

Because immersion of the gel pack in a water bath may prove inconvenient for some users, further experimentation suggested that a water-absorbent material, such as a foam sponge, could be used in place of a water bath in a manner that was more convenient for some users. For example, under experimental conditions, a layer of foam sponge, prepared from a consumer-grade household cleaning sponge, that was roughly ½″ thick and was wide and long enough to cover an eye mask shaped gel pack, was semi-saturated with water. When the gel pack was placed on a microwave turntable and this layer of wet foam sponge was placed directly on top of and covering the gel pack, and the sponge and gel pack were microwave-activated together, the heating of the gel pack was significantly more even than had been seen with microwave activation of the gel pack by itself, and the hot spots were nearly eliminated. While the sponge also heated up during use, it was quickly cooled down by running it under cold water, allowing it to be ready for the next such use. Of course other sizes and thicknesses of the water-absorbent material may be used so long as the gel pack is sufficiently covered.

The use of a water-absorbent material, such as a foam (including a foam sponge), which is used during the microwave-activation stage of heating of a thermal pack, in order to modulate the microwave activation of the thermal pack and produce a more even heating effect without hot spots, is not known in the art. While a foam sponge was used in experimentation, other water-absorbent or water-containing materials, including but not limited to woven and non-woven fabrics, hydrogels, and the like, could also be used.

Thus, in certain embodiments, either a volume of water which is deep enough to completely cover the gel pack, or a water-absorbent material that is capable of absorbing around 50 cc or more of water, and shaped and sized to cover a gel pack, can be used as a thermal regulator during microwave activation of the gel pack for use as a hot compress.

During experimentation, a method in which the gel was more rapidly and repeatedly pressed back and forth between the two sides of the gel pack also achieved a suitable redistribution of hotter and cooler gel, producing a more homogeneously-warmed gel pack. In this preferred method of preparing a gel pack for use, the user puts the gel pack on a surface (preferably a hard surface), preferably places a towel on the gel pack (to prevent burns from the hot spots), and presses with his/her palms alternately on one side and then the other of the pack (and not with both hands at once), pressing the gel all the way down to the surface, approximately 30 times back and forth over a duration of approximately 30 seconds.

Another exemplary method of using a compress assembly and kit will now be described. For the particular application of hot compress assembly, a gel pack and moistened sheet may be microwaved together or the gel pack may be microwaved on its own. If microwaved together, the microwave-activated wet sheet heats up, thereby providing an immediate application of moist heat, while the gel pack's heat gain services as a reservoir of continual heat generation which improves the duration of moist heat therapy. As mentioned above, the presence of a wet sheet on the gel pack can reduce some of the problems of uneven gel pack heating associated with microwave ovens. Once the components are placed on the face in position against the eye region, the compressive effect of the compress assembly or system may then be adjusted. In the illustrated example, this effect is augmented by tension exerted through an elastic strap that is attached to the gel pack and that goes around the side and back of the user's head.

In use, the user is free to manipulate the gel pack so as to conform to the user's particular anatomy, which allows the user to more conveniently and directly manipulate the gel and achieve anatomic conformation. Once the gel is manipulated into the desired conformation, the user may again adjust the compressive force of the frame by modifying the tension in the head strap. After use, the sheet can be disposed or can be used to clean or wipe the user's face and then disposed.

The compress devices, assemblies, kits and methods can be used for a variety of conditions and purposes. In the example of ocular discomfort, hot compress assembly can be used for various eye conditions including certain types of dry eye syndrome such as, for example, meibomian gland disease and other forms of blepharitis; “styes” (hordeola and chalazia); orbital and preseptal cellulitis; acute dacryocystitis; and other conditions. Hot compresses to the eyelids and periorbita can also used for certain postsurgical states, for the promotion of feelings of relaxation, for certain cosmetic or dermatological treatments, and for various other reasons. Cold or cool compress assemblies can be used for postoperative states following periorbital, intraorbital, or eyelid surgery; for symptomatic relief of irritating conditions such as acute allergic or viral conjunctivitis; for relief of migraines; to promote feelings of relaxation; to allow the application of topical skin therapies for cosmetic and dermatologic treatments, and for various other reasons.

With respect to other anatomical regions, the following exemplary conditions can be treated. Postsurgical and post-traumatic states of any body region, including strains, sprains, bruises and lacerations would be amenable to either hot or cold therapy, depending on physician instruction, the stage of recovery, and the type of fluid impregnated in the disposable sheet. Skin disorders of any region, such as dermatitis, impetigo, cellulitis, Stevens-Johnson syndrome, and others could be treated (as ancillary therapy to systemic medications) using medicated sheets and a physician-directed thermal application. Excessive muscular tension, for example in the angle of the jaw and in the paraspinal muscles of the cervical and lumbosacral regions, could be amenable to either hot or cold compress therapy. Joint disorders such as temporomandibular joint syndrome, arthritis, and tendinitis could be treated. These and other joint disorders of the angle of the jaw, the ankle, the knee, and the shoulder could be treated with cold or hot compress therapy. Postpartum states affecting the perineum could be treated with either cold or hot compress therapy. Unique conditions of the back and neck, such as herniated disks and postinjection conditions (e.g. from lumbar epidural administration) could be treated with cold or hot compress therapy. Frostbite of extremities such as the nose and ears could be treated with cool, warm, or hot compress therapy depending on the stage of recovery.

### EXAMPLES

### Example 1

### The Following Example Compares Dry Sheets with Wet Sheets

The thermal effects of a wet non-woven sheet on a heated gel pack were compared to the thermal effects of a dry non-woven sheet; these effects were tracked over time, and were also compared to the temperature of the gel pack itself.

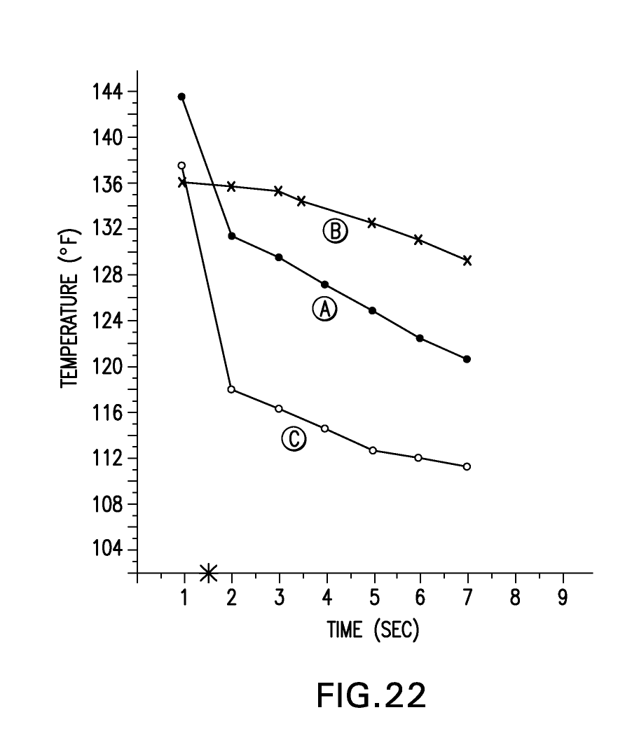

In this experiment, a single gel pack was heated with microwave activation. Temperatures on each of three different areas (each less than 1″ from the next area) on the surface of the gel pack were measured using three different thermometers placed in stable position. Once the temperatures at each of these areas achieved a maximum level, the temperature was recorded; and then various interventions were made (that is, a wet non-woven sheet was placed under the first thermometer tip; no intervention was made with the second thermometer tip; and a dry non-woven sheet was placed under the third thermometer tip). The temperature of each thermometer was then recorded at one-minute intervals. All temperatures are in ° F. The results are shown in FIG. 22 and Table I. It should be noted that the asterisk in FIG. 22 indicates when the dry non-woven sheet or wet non-woven sheet was applied to the gel pack.

TABLE I

| | A* | B* | C* |
|---|---|---|---|
| | 143.5 | 136.0 | 137.5 |
| 0.50 min | Wet NW | (Nothing) | Dry NW |
| 1.00 min | 131.5 | 135.9 | 118.0 |
| 2.00 min | 129.5 | 135.8 | 116.6 |
| 3.00 min | 127.4 | 134.6 | 114.6 |
| 4.00 min | 124.7 | 132.4 | 112.8 |
| 5.00 min | 122.5 | 131.2 | 112.1 |
| 6.00 min | 120.5 | 129.4 | 111.9 |
| Initial drop in temperature: | 12.0 | 0.1 | 19.5 |
| Subsequent drop: | 11.0 | 6.5 | 6.1 |
| Total drop: | 22.0 | 6.6 | 25.6 |

*The following interventions were made 30 seconds following the initial maximum temperature reading:
A: “Wet NW” = Wet non-woven sheet placed under thermometer tip.
B: The thermometer tip was allowed to stay in contact with the gel pack without any non-woven sheet placed beneath the thermometer and thus this served as a control.
C: “Dry NW” = Dry non-woven sheet placed under thermometer tip.

This experiment showed that a wet non-woven sheet allows more heat conductivity than a dry non-woven sheet. The initial drop in temperature with the dry sheet was 19.5 degrees, owing to the thermal insulation effect of the sheet itself. With a wet sheet, the insulation effect produced only a 12.0 degree drop in temperature. With the wet sheet, the sheet itself still served as a partial insulator, but the presence of water produced a conductive effect that counteracted this insulation effect.

Following the initial drops in temperature for both the wet and dry sheets, the wet sheet did undergo a more rapid decline in temperature relative to the more gradual and steady decline seen with the dry sheet. It is believed that this more rapid subsequent temperature decline of the wet sheet under laboratory benchtop conditions was actually due to greater heat conductivity from the heated wet sheet into the surrounding air. Because, during actual use, the sheet will be in direct contact with the skin of the user, such heat loss would be of benefit to the user, as the heat loss would be into the user's skin rather than into the surrounding air.

### Example 2

### The Following Experiments Compares the Subjective Effect of a Dry Non-Woven Sheet and a Moistened Non-Woven Sheet

The following experiment was performed to test the effect of moistening a dry non-woven sheet used under a heated gel pack. An eye mask shaped gel pack was heated using microwave activation, to a temperature of around 125 to 135° F. Under experimental conditions, a kit comprising the heated gel pack and a dry non-woven sheet shaped as in FIG. 11 was placed on the periocular and periorbital regions of a user, with the dry non-woven sheet in contact with the user's skin and the gel pack positioned directly on top of the dry non-woven sheet. The user's subjective experience was recorded.

The kit comprising the gel pack and dry non-woven sheet unit was then removed from the user's ocular and periorbital regions. A wet sponge was touched to the dry non-woven sheet in order to moderately dampen the surface of the non-woven sheet. Then, the kit comprising the gel pack and damp non-woven sheet were placed on the user's ocular and periorbital regions, with the damp non-woven sheet in contact with the user's skin and the gel pack directly on top of the non-woven sheet. The user's subjective experience was again recorded.

### Results:

With the dry non-woven sheet, the user's sense was that the warmth of the gel pack was barely appreciated. The user did not appreciate the thermal effect desirable in a hot ocular compress.

Once the non-woven sheet was dampened and the kit was reapplied, the user immediately appreciated a very significant thermal effect. The user felt that this temperature was extremely effective as a therapeutic hot compress for the ocular and periorbital regions. This effect was felt to be much more significant than is accounted for in the objective measurements made to compare wet and dry sheets in contact with the gel pack.

### Example 3

### The Following Example Compares the Use of Non-Woven Fabric Sheets with Paper Towels

For the paper towels, the towels were taken from a roll of Bounty® two-ply White paper towels, marked “KEEPS WORKING” and “THICK AND DURABLE,” with sheets 11″×11,″ with 94 sheets per roll. The non-woven sheets used were made of polymer and pulp, as previously described. Sheets were either cut to the size and contour shown in the illustrated embodiment of FIG. 11, or cut to smaller sizes as needed.

1. The Water Bath Test

To simulate the condition of the pre-moistened preparation of a sheet, compress-shaped sheets were immersed in a shallow water bath for 10 minutes and then removed. It was discovered that the 2-ply structure of the paper towel sheet had unexpectedly come apart, separating into two single-ply sheets as it was being lifted from the water bath. Upon further testing, it was found that separation of the plied paper towel sheets occurred after as little as 15 seconds of immersion in the shallow water bath.

Because this discovery suggested that long-term storage of a pre-moistened paper towel would best be performed using a single-ply paper towel, the paper towels were separated into single ply, and testing was performed on these.

1A. The Water Absorption Test (on a 1-Ply Paper Towel)

First, the amount of water absorption of each sheet was measured as follows:

3 ml (3.0 g) of water was placed in the center of a scale. Next, a 2″×3″ sheet of each material was used to absorb the water from the scale so that the sheet was saturated. The sheet was then held up by one corner and allowed to drip gently onto the scale (splashing was avoided) until the time between the drips exceeded 5 seconds. The residual weight of the water remaining on the scale was then recorded. The weight of absorbed water in the sheet was calculated by subtracting the residual weight of water on the scale from the initial weight of the water (3.0 g). The water capacity per square inch of each sheet type was then calculated. The potential water capacity of a full eye compress sheet (around 26.9 square inches) was then calculated and the results are shown in Table II.

TABLE II

| | Non-woven | | 1-Ply Paper | |
|---|---|---|---|---|
| | 2″ × 3″ | | Towel 3″ × 3″ | |
| Trial# | Residual Water | | Residual Water | |
| 1 | 0.3 | g | 2.2 | g |
| 2 | 0.3 | g | 2.3 | g |
| 3 | 0.3 | g | 2.3 | g |
| 4 | 0.3 | g | 2.2 | g |
| Avg residual: | 0.3 | g | 2.25 | g |
| Avg capacity: | 2.7 | g | 0.75 | g |
| # Sq in. | 6 | sq in | 9 | sq in |
| Capacity/sq in: | 0.45 | g/sq in | 0.08 | g/sq in |
| Capacity: full sheet | 12.11 | g | 2.24 | g |

It was concluded that, given equivalent dimensions of non-woven and single-ply paper towel sheets, the non-woven sheet would hold over 5 times as much moisture as a single-ply paper towel sheet. This was an unexpected result because paper towel sheets are marketed as being especially water-absorbent and good for cleaning up spills, whereas non-woven sheets are promoted for their dirt-cleaning behavior.

1B. The Wear-and-Tear Test (on a 1-Ply Paper Towel)

The resistance to tearing during buttoning and removal was then tested. Sheets were tested by buttoning them onto a external frame and then removing them 10 times. Prior to such testing, two buttonholes were cut as 0.625″ slit in the paper towel and non-woven sheets as 0.625″ slits each. The results are shown in Table III.

TABLE III

| Sheet Type | Buttonhole widths after 10 buttonings |
|---|---|
| Non-woven, Dry | 0.625″ and 0.625″ |
| Non-woven, Wet | 0.625″ and 0.625″ |
| 1-Ply Paper Towel, Dry | 0.625″ and 0.750″ |
| 1-Ply Paper Towel, Wet | One buttonhole tore open completely |
| | (through the top of the sheet). |
| | The other buttonhole tore open |
| | to 1.5″ |

This test demonstrated the significant inferiority of a single-ply paper towel to routine use and manipulation in a preferred embodiment of the present invention where a wet sheet is attached to an external frame via buttons. This was an unexpected result because higher-quality paper towels made for cleanup rather than just for spills alone would be expected to withstand a moderate amount of wear and tear while wet.

1C. The Drying Time Test (on a 1-Ply Paper Towel)

During testing of the function of a single-ply paper towel, it was casually noted that the drying time of the single-ply paper towel sheet was quite rapid compared to a single-ply non-woven sheet. This was an unexpected result because, as noted above, paper towel sheets are marketed as being especially water-absorbent and good for cleaning up spills, whereas non-woven sheets are promoted for other reasons such as dirt cleanup.

Drying time of the different materials was then tested. This was done by placing 3 drops on a 3″×3″ sheet of each material and measuring the diameter of the wet or damp area at successive time periods. It was immediately noted that the drops placed on the paper towel sheet tended to rapidly become absorbed into the sheet, whereupon the area of wetness expanded rapidly (presumably by capillary action of the paper towel). In contrast the, drops placed on the non-woven sheet tended to bead up initially and were more slowly absorbed into the sheet; and the area of wetness did not tend to expand, but rather to stay in a small-diameter configuration. The results are shown in Table IV.

TABLE IV

| Time | Non-woven Sheet Diameter | Single Ply Paper Towel Diameter |
|---|---|---|
| 6:45 am | 0.625″ | 1.5″ |
| 6:50 am | 0.625″ | 2.5″ |
| 6:55 am | 0.625″ | 2.25″ (barely damp) |
| 7:00 am | 0.625″ | Completely dry |
| 7:05 am | 0.625″ | |
| 7:10 am | 0.75″ | |

(several measurements deleted for brevity's sake)

| | |
|---|---|
| 7:45 am | 1″ × 1″ |

Testing was stopped at this point, after it became clear that, given a set amount of moisture, the non-woven sheet retained such moisture far longer than the paper towel. The rapidity of drying in the paper towel was possibly due to improved capillary action and enhanced spread of moisture along the sheet. This markedly greater retention time of moisture by a non-woven sheet was unexpected given that paper towel sheets are marketed as being especially water-absorbent and good for cleaning up spills, whereas non-woven sheets are promoted for their dirt-cleaning behavior. Because it is anticipated that users may prefer to re-use sheets during a particular eye compress treatment session (which may last 20 minutes or more), and because it is also anticipated that users will subject moist sheets to microwave heating, which would increase evaporation rates, a more rapid-drying sheet is undesirable.

The above data show that a single-ply non-woven sheet can hold nearly 6 times as much water as a single-ply paper towel, can maintain equivalent amounts of moisture at least 4 to 6 times longer during working conditions, and maintains its integrity much better during routine handling. Taken together, these data suggest a strong disadvantage for choosing pre-moistened paper towels as compared to pre-moistened non-woven sheets for use in an eye compress device.

2A. The Water Absorption Test (for a 2-Ply Paper Towel)

The same procedures as set forth in Example 1A above were performed to gauge the water absorption of a 2-ply paper towel. The results are shown in Table V.

TABLE V

| | 2-Ply Paper | |
|---|---|---|
| | Towel 3″ × 3″ | |
| Trial# | Residual Water | |
| 1 | 0.5 | g |
| 2 | 0.5 | g |
| 3 | 0.5 | g |
| 4 | 0.5 | g |
| Avg residual: | 0.5 | g |
| Avg capacity: | 2.5 | g |
| # Sq in. | 9 | sq in |
| Capacity/sq in: | 0.28 | g/sq in |
| Capacity: full sheet | 7.47 | g |

Thus, a non-woven sheet would be expected to hold roughly 60% more moisture than a two-ply paper towel sheet (12.11 g vs. 7.47 g). This was an unexpected result because an intact two-ply paper towel sheet (unlike a pulled-apart one-ply paper towel sheet) would have been expected to be much more water-absorbent, consistent with marketing of paper towels for spill clean-ups, compared to non-woven sheets which are marketed for dirt cleanup.

2B. The Drying Time Test (on a 2-Ply Paper Towel)

The drying time of a two-ply paper towel sheet was then assessed. The same procedures as set forth in Example 1C above were performed to gauge the water absorption of a 2-ply paper towel. The results are shown in Table VI.

TABLE VI

| Time | | Double-Ply Paper Towel Diameter |
|---|---|---|
| 1:00 | pm | 1.625″ |
| 1:05 | | 2.25″ |
| 1:10 | | 2.25″ |
| 1:15 | | 2.0″ (faint/damp) |
| 1:20 | | 2.0″ (difficult to measure) |
| 1:25 | | 1.5″ (difficult to measure) |
| 1:30 | | Dry |

Thus, the double-ply paper towel sheet dried in less than half the time of a non-woven sheet holding an equivalent amount of water. This was an unexpected result because, again, the two-ply paper towel, now fully intact rather than pulled-apart into a single ply, still exhibited a very similar rapid drying-off, and poor long-term water retention, relative to an item (a non-woven sheet) that is sold primarily not for cleaning spills, but for holding on to dirt.

In conclusion, non-woven sheets were determined to be superior to both single- and double-ply paper towel sheets for pre-moistened use with a thermally-adjustable eye compress device.

2C. Weight-Based Drying Time Test on Human Using an Eye Compress Assembly (2-Ply Paper Towel vs. Single-Ply Nonwoven Sheet)

The drying time of a two-ply paper towel sheet was compared to that of a single-ply nonwoven fabric sheet during actual use on a human subject in the context of using a full eye compress assembly.

This experiment was performed using full-size sheets (one nonwoven, one paper towel) shaped as in FIG. 11. Dry weights were taken. The sheets were moistened using a spray bottle filled with tap water. Wet weights were taken. The sheets were applied to gel packs of 2.5 ounces which had been heated in a microwave oven and manipulated to achieve even homogeneous temperatures, and the gel pack-and-sheet assemblies were then applied to the face of a user. The temperature between the user's skin and the sheet was measured at the start and at the end of a 5-minute period. The final weights of the sheets were then taken. Moisture loss was calculated. The results are shown in Table VII

TABLE VII

| | Sheet Type | |
|---|---|---|
| | Nonwoven | Paper Towel |
| Dry Weight (oz.) | 0.06 | 0.05 |
| Starting Wet Weight (oz.) | 0.21 | 0.16 |
| Skin-Sheet Temp, at start, ° F. | 115 | 110 |
| Skin-Sheet Temp, after 5 mins, ° F. | 103 | 98 |
| Ending Wet Weight (oz.) | 0.18 | 0.11 |
| Weight Loss due to Evaporation (oz.) | 0.03 | 0.05 |
| Initial Weight of Water (calculated, oz.) | 0.15 | 0.11 |
| Percent Water Loss | 20% | 45% |

This experiment shows that, under experimental hot compress use on the human body, the percentage of water lost from the nonwoven sheet was less than half the percentage of water lost from the paper towel. The results are all the more remarkable when it is observed that the skin-sheet temperature was maintained at 5 degrees higher for the nonwoven sheet than for the paper towel sheet, a fact which would tend to encourage more water loss from the nonwoven sheet.

### Example 4

### The Following Example Compares the Use of Non-Woven Fabric Sheets with Terrycloth Towels

In this experiment, the thermal effects of a wet non-woven sheet and a wet terrycloth towel were tracked over time, and were also compared to the temperature changes of a gel pack.

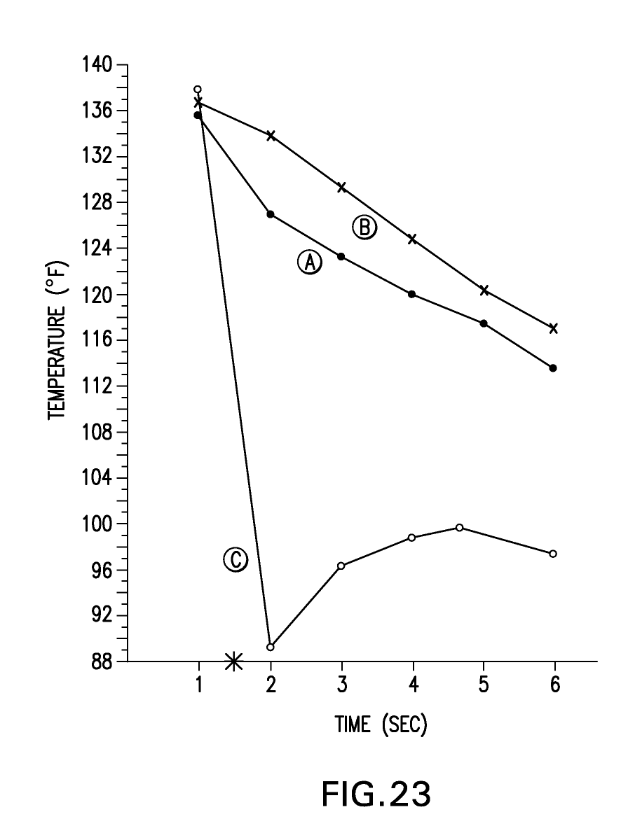

Specifically, a single gel pack was heated with microwave activation. Temperatures on each of three different areas on the surface of the gel pack (each less than 1″ from the next area) were measured using three different thermometers. Once the temperatures at each of these areas achieved a maximum level, the temperature was recorded; and then various interventions were made (that is, a wet non-woven sheet was placed under the first thermometer tip; no intervention was made with the second thermometer tip; and a wet terrycloth sheet was placed under the third thermometer tip). The temperature was then recorded at one-minute intervals. All temperatures are in ° F. FIG. 23 and Table VIII show the results. It should be noted that the asterisk in FIG. 23 indicates when the wet non-woven sheet or terrycloth towel was applied to the gel pack.

TABLE VIII

| | A* | B* | C* |
|---|---|---|---|
| | 135.9 | 136.6 | 137.8 |
| 0.50 min | Wet NW* | (Nothing) | Wet TC* |
| 1.00 min | 126.9 | 133.9 | 89.1 |
| 2.00 min | 123.6 | 129.6 | 96.2 |
| 3.00 min | 120.0 | 125.2 | 98.9 |
| 4.00 min | 117.8 | 120.2 | 99.1 |
| 5.00 min | 113.5 | 117.7 | 97.5 |
| Initial drop in temperature: | 9.0 | 2.7 | 48.7 |
| Subsequent drop: | 13.4 | 16.2 | (8.4) |
| Total drop: | 22.4 | 18.9 | 40.3 |

*The following interventions were made 30 seconds following the initial maximum temperature reading.
A: “Wet NW” = Wet non-woven sheet placed
B: No sheet was placed
C: “Wet TC” = Wet terrycloth towel placed

This experiment showed that the initial very large drop in temperature created by the thermal barrier effect of the wet terrycloth towel was sustained through time. Even though the initial temperature of the gel pack was quite hot (nearly 140° F.), the wet terrycloth towel blocked heat so significantly that the effective temperature at the surface of the terrycloth towel never reached the preferred minimum therapeutic window of 104° F., much less the estimated optimal therapeutic level of 120 to 125° F.

### Example 5

### The Following Example Shows how a Separate Wet and Dry Sheet or a Sheet with Wet and Dry Portions can be Used to Serve as a Thermal Reservoir for Certain Regions and Serve as a Thermal Barrier for Other Regions of the Body

To test the thermal effects of a combination of dry and wet sheets on the heat conductivity of the gel pack, two experiments were performed.

In the first experiment, a gel pack was heated; then a dry sheet was placed over the gel pack; and then a wet sheet was interposed between the gel pack and the dry sheet. The purpose of this experiment was three-fold: (1) to see what effect a dry sheet (preferred by some users during hot compress therapy) would have on heat conductivity compared to the gel pack alone; and (2) to see whether a wet sheet placed underneath the dry sheet would increase the heat intensity transmitted to the user's anatomy.

In the second experiment, a gel pack was heated; then a bilayer of a wet and dry sheet were placed over the gel pack (wet sheet against the gel pack); and then the dry sheet was removed.

In both tests, a rapid-read digital kitchen thermometer (Polder) was used. Because it had been found that such a thermometer will read a higher temperature when pressed more firmly into a heated gel pack, care was taken to lay the thermometer horizontally, with its sensor tip resting gently on the surface of the gel pack with no excessive downward pressure. Care was also taken to assure that the sensor tip remained in exactly the same position in relation to the surface of the gel pack (e.g., lying in a particular small declivity). This was necessary because it had been found that the surface temperature of a microwave-heated gel pack can vary greatly from one area to another.

The temperature was not recorded until it had reached a relatively stable level, which was determined by absence of temperature change within an interval of about 3 seconds.

The gel packs used were based on the design in FIG. 3A. All sheets were made of non-woven material as described previously, and were based on the design in FIG. 11. All wet sheets were saturated with about 9 cc of tap water. The results are shown in Table IX.

TABLE IX

| | Trial#: | | |
|---|---|---|---|
| First Experiment | 1 | 2 | 3 |
| Gel pack only | 142 | 140 | 144 |
| Gel pack + dry sheet | 121 | 121 | 122 |
| Gel pack + Wet sheet + Dry sheet* | 127 | 126 | 128 |

*The temperature first dipped and then rose; the values shown are the maximum temperature following the rise.

This data clearly confirm the heat conductivity effect of a wet sheet as compared to a dry sheet, because the addition of a wet sheet against the gel pack increased the temperature of the dry sheet.

To show that the addition of a dry sheet on top of a wet sheet will serve as an effective thermal barrier a second experiment was run as described above. The results are shown in Table X.

TABLE X

| | Trial#: | | | |
|---|---|---|---|---|
| Second Experiment | 1 | 2 | 3 | 4 |
| Gel pack only | 144 | 144 | 122 | 145 |
| Gel pack + Wet sheet + Dry sheet* | 131 | 128 | 115 | 130 |
| Gel pack + Wet sheet | 134 | 132 | 118 | 133 |
| Gel pack only | 128 | 125 | 116 | 126 |

*The temperature first dipped and then rose; the values shown are the maximum temperature following the rise.

These data show that the dry sheet does serve as a thermal barrier when placed on top of a wet sheet. They also show that a room-temperature wet sheet acquires and stores heat from a heated gel pack, thus serving as a heat source independent of the gel pack.

Thus, the following conclusions can be drawn:

(1) the dry sheet did provide a measurable thermal barrier effect when used on top of the wet sheet;
(2) the wet sheet aided the thermal conductivity of heat from the gel pack,
(3) the wet sheet, once it had acquired heat from the gel pack, served as a source of heat that was independent from that of the gel pack;
(4) the wet sheet provided a thermal barrier effect (thus yielding a surface temperature that was not as intense as the surface of the gel pack itself, but which was higher than the temperature of the dry sheet).

### Example 6

### The Following Example Shows how to Regulate the Temperature of a Gel Pack to Avoid Hot Spots in the Gel Pack

To test the evenness of heating of an exemplary eye mask shaped gel pack, a gel pack was activated in a microwave under various conditions. In all experiments, temperatures listed are in ° F.; all gel pack temperatures were taken on the surface of the gel pack (by laying a Polder kitchen thermometer on the surface of the gel pack and indenting slightly); all gel packs weighed 2.5 ounces; and the pre-activation temperature of the gel packs was 71° F. The temperature of the gel packs were measured in 3 areas (left, center, and right) in order to compare the activated temperature at each location and to check for hot spots.

### First Experiment: Dry Gel Pack

For the first experiment, the gel pack was placed on the central turntable of a 1,000 watt microwave and the microwave was activated on a “high” setting for specified amounts of time. The results are shown in Table XI.

TABLE XI

| | Trial#: | | |
|---|---|---|---|
| | 1 | 2 | 3 |
| Activation time, secs | 30 | 30 | 26 |
| Gel pack, left (° F.) | 108 | 137 | 165 |
| Gel pack, center (° F.) | 142 | 143 | 145 |
| Gel pack, right (° F.) | 134 | 139 | 113 |

The first experiment showed the unpredictable uneven heating of the gel pack after microwave activation.

### Second Experiment: Water Bath

In this experiment, the gel pack was placed in a shallow plastic container measuring about 8″×5″×1″. Cold tap water was added to the container. In the first trial, the gel pack was allowed to float to the surface such that the top and central portion of the gel pack was exposed to the air. Because this exposed portion proved to be a hot spot following microwave activation, in the subsequent trials, the gel pack was weighted down at both ends such that it was entirely submerged under the surface of the water. In addition to measurements of the gel pack surface temperature, measurements of the water bath temperature were also taken. The results are shown in Table XII.

TABLE XII

| | Trial#: | | | | |
|---|---|---|---|---|---|
| | 1 | 2 | 3 | 4 | 5 |
| Water cc | 200 | 300 | 300 | 300 | 300 |
| Initial water temp (° F.) | 62 | 62 | 61 | 59 | 58 |
| Extent of coverage of pack* | (a) | (b) | (b) | (b) | (b) |
| Activation time, secs | 90 | 90 | 120 | 150 | 180 |
| Gel pack, left | 110 | 109 | 112 | 123 | 143 |
| Gel pack, center | 148 | 120 | 118 | 126 | 145 |
| Gel pack, right | 122 | 105 | 119 | 129 | 145 |
| Water bath, left | 120 | 110 | 124 | 133 | 137 |
| Water bath, center | 122 | 108 | 124 | 132 | 137 |
| Water bath, right | 120 | 105 | 123 | 132 | 136 |

(a): slightly exposed (floating gel pack)
(b): completely submerged; weighted down at both sides to the bottom of the container

The second experiment showed that the gel pack could achieve significantly more even temperatures, more predictable results, and an absence of hot spots, when submerged in a water bath and then microwave-activated, than when exposed to air and then microwave-activated. The second experiment also showed that the water bath did not get hot enough to account for the immediate increase in temperature of the gel pack. Thus, the gel pack received most of its increase in heat from direct microwave activation of the gel contents.

### Third Experiment: Damp Sponge

In this experiment, consumer-grade cleaning sponges (O-Cel-O brand, measuring 7.7″×4.2″×1.5″ and packaged individually, with each sponge sold slightly premoistened within the package) were prepared to serve as thick layers covering the gel pack. Prior to gel pack activation, the gel pack was placed on the center of a rotating microwave turntable. The prepared sponge layer was then placed on top of the gel pack, completely covering it. The microwave was then activated for specified time periods. The results are shown in Table XIII.

TABLE XIII

| | Trial#: | | | | |
|---|---|---|---|---|---|
| | 1 | 2 | 3 | 4 | 5 |
| Sponge preparation* | (a) | (b) | (c) | (d) | (e) |
| Activation time, secs | 35 | 35 | 40 | 45 | 50 |
| Gel pack, left | 136 | 103 | 137 | 128 | 136 |
| Gel pack, center | 136 | 104 | 143 | 129 | 118 |
| Gel pack, right | 99 | 112 | 130 | 115 | 130 |

*Sponge preparation:
(a) Full sponge was taken from package and placed on top of gel pack.
(b) Full sponge was taken from package, 40 cc water was added to one side of sponge, and sponge was placed (wet side down) on top of gel pack.
(c) A sponge was taken from package. The sponge was sliced horizontally such that a thin irregular section ¼-½″ was produced. This thin layer was saturated under running tap water, and then was partly wrung out. The sponge was placed on the gel pack.
(d) A sponge was taken from its package. A thin layer, ½ to ¾″ thick, and more regular in contour than (c), was prepared. 80 cc of water was added to one side of this layer of sponge. The layer was placed wet side down on top of the gel pack.
(e) The same sponge as used in (d), already heated in the microwave during a prior experiment, was rinsed under cold tap water until it was no longer warm to the touch. 80 cc of water was added to one side of this layer of sponge. The layer was placed wet side down on top of the gel pack.

(e) The same sponge as used in (d), already heated in the microwave during a prior experiment, was rinsed under cold tap water until it was no longer warm to the touch. 80 cc of water was added to one side of this layer of sponge. The layer was placed wet side down on top of the gel pack.

The third experiment showed that an experimental prototype of a layer of wettable material can be used to regulate the microwave activation of a gel pack and produce heating that is nearly as even, with the virtual elimination of hot spots, as a water bath used for the same purpose.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. An eye compress kit comprising:
a compress assembly comprising:
a thermally adjustable gel pack configured to be applied against an eye region of a user's body, the gel pack comprising a casing having a top portion, a bottom portion, a right portion and a left portion, the casing defining a chamber holding a thermally activatable gelatinous substance; and
a strap attached to the casing to secure the gel pack against the user's eye region and to exert compressive forces to the gel pack; and
a plurality of moistened, disposable fibrous non-woven fabric sheets adapted to be positioned between the gel pack and the user's eye region wherein the fabric sheets are removable from the outer surface of the gel pack.

2. The eye compress kit of claim 1, wherein the eye region is the periocular region.

3. The eye compress kit of claim 1, wherein the eye region is the periorbital region.

4. The eye compress kit of claim 1, wherein the eye region is both the periorbital region and the periocular region.

5. The eye compress kit of claim 1, wherein the eye region includes the temples of the head.

6. The eye compress kit of claim 1, wherein the top portion of the gel pack comprises a top lip, the top lip having a height sufficient to accommodate at least one fastener to attach the gel pack to an external frame.

7. The eye compress kit of claim 1, wherein each of the plurality of sheets has a top portion configured to accommodate at least one fastener to attach each of the plurality of sheets to an external frame.

8. The eye compress kit of claim 6, wherein the top lip of the gel pack comprises at least one aperture for receiving a fastener.

9. The eye compress kit of claim 7, wherein the top portion of each of the plurality of sheets comprises at least one aperture for receiving a fastener.

10. The eye compress kit of claim 6, wherein the top lip of the gel pack comprises at least one fastener.

11. The eye compress kit of claim 7, wherein the top portion of each of the plurality of sheets comprises at least one fastener.

12. The eye compress kit of claim 1, wherein the strap is an adjustable strap.

13. The eye compress kit of claim 1, wherein the strap is fabricated from a material different than the material of the casing.

14. The eye compress kit of claim 1, wherein the gel pack has a periphery defining a left lip and a right lip and the strap is attached to the left lip and the right lip.

15. The eye compress kit of claim 1 wherein the gel pack is in the form of a mask, the mask comprising a left section and a right section, the left section configured to be applied against the left periocular and periorbital region of the user and the right section configured to be applied against the right periocular and periorbital region of the user, the mask comprising at least two layers of flexible sheets sealed about their peripheries to form the chamber.

16. The eye compress kit of claim 15, wherein the mask comprises a bottom portion shaped like a bell curve creating a notch to accept the nasal wings of the user.

17. The eye compress kit of claim 15, further comprising a left wing configured to be applied against the left temple of the patient and a right wing configured to be applied against the right temple of the patient.

18. The eye compress kit of claim 1, wherein the gel pack is in the form of an eye patch configured to be applied against either the left or the right eye region of the user.

19. The eye compress kit of claim 1, wherein the casing comprises a non-vinyl material.

20. The eye compress kit of claim 1, wherein the volume and viscosity of the gelatinous substance are such that a user can manipulate the gelatinous substance to maintain its position in a specific area of the eye region for a period of at least 5 minutes by applying topical pressure to the gel pack.

21. The eye compress kit of claim 1, further comprising at least one separate fastener that is not integral with either each of the plurality of sheets or the gel pack.

22. The eye compress kit of claim 1, wherein each of the plurality of sheets is pre-moistened and contained within a dispenser.

23. The eye compress kit of claim 1, wherein each of the plurality of sheets has a surface area greater than the surface area of the gel pack such that in use, each sheet extends beyond the edges of the gel pack on all sides so that the user's face is contacted in all applied areas by the sheet rather than by the gel pack directly.

24. The eye compress kit of claim 1, further comprising an external frame comprising a frame body having a top portion, a bottom portion and side portions, the external frame positionable against to the outwardly facing surface of the gel pack, the external frame fabricated from a material stiff enough to support at least a portion of the gravitational weight of the gel pack such that when the external frame is in a vertical orientation it does not buckle when the gel pack is attached thereto and secured against the eye region of the user.

25. The eye compress kit of claim 24, wherein the top portion and at least one of the side portions or the bottom portion of the external frame is attachable to the gel pack.

26. The eye compress kit of claim 2, wherein each of the plurality of non-woven fabric sheets has a greater surface area that the gel pack and the gel pack has a greater surface than the external frame.

27. An eye compress kit comprising:
a thermally adjustable gel pack configured to be applied against the eye region of a user's body, the gel pack comprising a casing having a top portion, a bottom portion, a right portion and a left portion, the casing defining a chamber holding a thermally activatable gelatinous substance;
a frame assembly comprising:
an external frame comprising a frame body having a top portion, a bottom portion and side portions, the external frame positionable against the outwardly facing surface of the gel pack; and
a strap attached to the external frame; and
a plurality of moistened, disposable fibrous non-woven fabric sheets adapted to be positioned between the gel pack and the user's eye region wherein the fabric sheets are removable from the outer surface of the gel pack.

28. The eye compress kit of claim 27, wherein at least the top portion of the external frame comprises at least one fastener to attach the gel pack and one or more of the plurality of sheets to the external frame.

29. The eye compress kit of claim 28, wherein the at least one fastener is selected from the group consisting of a male/female fastener, a magnet, VELCRO or a string.

30. The eye compress kit of claim 26, wherein the kit further comprises at least one separate fastener that is separable from the external frame.

31. The eye compress kit of claim 27, wherein the external frame is fabricated from a material stiff enough to support at least a portion of the gravitational weight of the gel pack such that when the external frame is in a vertical orientation it does not buckle when the gel pack is attached thereto and secured against the eye region of the user.

32. The eye compress kit of claim 27, wherein the external frame comprises a left section whose internal periphery defines a left opening and a right section whose internal periphery defines a right opening, both the left and right openings sized to allow the user to manipulate the position of the gelatinous substance in the gel mask by applying topical pressure to the gelatinous substance.

33. The eye compress kit of claim 27, wherein the internal periphery of the frame body defines a single aperture sized to allow the user to manipulate the position of the gelatinous substance in the gel mask by applying topical pressure to the gelatinous material.

34. The eye compress kit of claim 27, wherein a first button and a second button are attached to the top portion of the frame body and, in use, the first and second button are received by a first and second opening of the casing and a first and second opening of at least one of the plurality of sheets, the first openings and the second openings aligned with the respective first and second buttons of the external frame to secure the gel pack and the at least one of the plurality of sheets to the external frame.

35. The eye compress kit of claim 27, wherein the top portion of the external frame comprises at least one fastener to secure the gel pack and at least one of the plurality of sheets to the external frame and the left and right side portions of the external frame are attached to the strap such that tightening or loosening of the strap exerts a controllable horizontal pressure on the external frame, such pressure being largely independent from the vertical support provided by the external frame to the gel pack via at the least one fastener during use.

36. The eye compress kit of claim 35, where the at least one fastener is a pair of fasteners spaced apart from each other.

37. The eye compress kit of claim 27, wherein the frame body comprises a bridging portion that bridges the top portion and the bottom portion of the frame body.

38. The eye compress kit of claim 37, further comprising a gel impressor comprising a flexible bending strip configured to be placed in contact with the back surface of the bridging portion of the frame body of the external frame.

39. An eye compress kit comprising:
a compress assembly comprising:
a gel pack configured to be applied against the eye region of a user's body, the gel pack comprising a casing having a top portion, a bottom portion, a right portion and a left portion, the casing defining a chamber holding a thermally activatable gelatinous substance; and
a strap attached to the casing to secure the gel pack against the user's eye region; and
a plurality of moistened, disposable foam sheets adapted to be positioned between the gel pack and the eye region of the user, wherein the foam sheets are removable from the outer surface of the gel pack.

40. An eye compress kit comprising:
a compress assembly comprising:
a thermally activatable gel pack configured to be applied against the eye region of a user's body, the gel pack comprising a casing having a top portion, a bottom portion, a right portion and a left portion, the casing defining a chamber holding a thermally activatable gelatinous substance; and
a strap attached to the casing to secure the gel pack against the user's eye region and to exert compressive force to the gel pack; and
a plurality of moistened, disposable film sheets adapted to be positioned between the gel pack and the eye region of the user, wherein the film sheets are removable from the outer surface of the gel pack.

41. An eye compress kit comprising:
a compress assembly comprising:
a thermally adjustable gel pack configured to be applied against the eye region of a user's body, the gel pack comprising a casing having a top portion, a bottom portion, a right portion and a left portion, the casing defining a chamber holding a thermally activatable gelatinous substance; and
a strap attached to the casing to secure the gel pack against the user's eye region and to exert compressive forces to the gel pack; and
a plurality of disposable sheets adapted to be positioned between the gel pack and the user's eye region wherein the sheets are removable from the outer surface of the gel pack, wherein either:
some of the plurality of sheets are moistened and others of the plurality of sheets are dry; or
a portion of each of the plurality of sheets is moistened and another portion of each of the plurality of sheets is dry, wherein the dry sheets or the dry portion of the sheets define apertures to allow transmission of heat from the wet sheet to a selective portion of the user's eye region.

42. An eye compress kit comprising:
a compress assembly comprising:
a thermally adjustable gel pack configured to be applied against the eye region of a user's body, the gel pack comprising a casing having a top portion, a bottom portion, a right portion and a left portion, the casing defining a chamber holding a thermally activatable gelatinous substance; and
a strap attached to the casing to secure the gel pack against the user's eye region and to exert compressive forces to the gel pack;
a plurality of moistened disposable sheets wherein the sheets are removable from the outer surface of the gel pack; and
a non-disposable dry layer defining apertures, the dry layer positioned between either the gel pack and at least one of the plurality of sheets during use or positioned between at least one of the plurality of sheets and the user's face during use, wherein the apertures allow transmission of heat from the wet sheet to a selective portion of the user's eye region during use.

43. A method of using the eye compress kit of claim 1, comprising:
providing the gel pack;
heating or cooling the gel pack;
providing at least one of the plurality of sheets; and
placing at least one of the plurality of sheets between the gel pack and the eye region of the user.

44. A method of preparing an eye compress assembly for use comprising:
providing a thermally adjustable gel pack containing a thermally activatable gelatinous substance, the gel pack configured to be applied to an eye region of a patient;
immersing the gel pack into a water bath so that the gel pack is completely covered by the water in the bath; and
microwaving the water bath containing the gel pack.

45. A method of preparing an eye compress assembly for use comprising:
providing a thermally adjustable gel pack containing a thermally activatable gelatinous substance, the gel pack configured to be applied to an eye region of a patient, the gel pack having an outer surface;
applying a fluid-absorbent material to the outer surface of the gel pack, the fluid-absorbent material at least semi-saturated with a fluid; and
microwaving the gel pack and the fluid-absorbent material.

46. A method of preparing an eye compress assembly for use comprising:
providing a thermally adjustable gel pack containing a thermally activatable gelatinous substance, the gel pack configured to be applied to an eye region of a patient, the gel pack having an outer surface and a left and a right side;
placing the gel pack on a surface; and
alternatively pressing the left and right side of the gel pack all the way down to the surface approximately 30 times back and forth over a duration of approximately 30 seconds.
